(19) United States
(12) Patent Application Publication
(10) Pub. No.: US 2010/0190645 A1
(43) Pub. Date: Jul. 29, 2010

(54) SYNERGISTIC FUNGICIDAL ACTIVE COMPOUND COMBINATIONS COMPRISING FORMONONETIN

(76) Inventors: Anne Suty-Heinze, Langenfeld (DE); Darren Mansfield, Kurten (DE); Stéphanie Gary, Lyon (FR)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/521,874

(22) PCT Filed: Jan. 19, 2008
(86) PCT No.: PCT/EP2008/000400

§ 371 Date: Feb. 9, 2010

(30) Foreign Application Priority Data
Feb. 2, 2007 (EP) 07002362.7

(51) Int. Cl. A01C 1/06; A61K 31/352; A01N 43/653

(52) U.S. Cl. 504/100; 514/456; 514/383

(57) ABSTRACT

Novel active compound combinations comprising formononetin of formula (I)

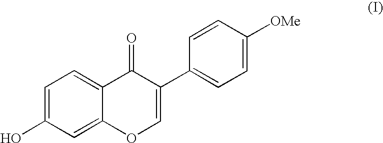

and the active compound groups (2) to (24) listed in the description have very good fungicidal properties.

The present invention relates to novel active compound combinations comprising firstly formononetin and secondly further known fungicidally active compounds, which novel active compound combinations are highly suitable for controlling unwanted phytopathogenic fungi.

Formononetin of formula (I)

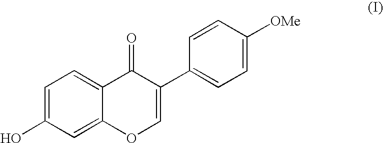

is an isoflavone, which is produced by red clover (Trifolium pratens), and is known to enhance the root growth of agricultural crops (U.S. Pat. No. 5,002,603) by stimulating the growth of rhizobia.

It has now been discovered that formononetin is also a potent enhancer of the activity of fungicides. This enhancing effect is overadditive. This means that the fungicidal activity of the mixtures of formononetin and the fungicides is higher than the sum of the fungicidal activities of the components alone.

Besides Formononetin, also salts of formononetin can be mixed with active compounds, selected from groups (2) to (24), to give fungicidal mixtures with synergistic activities. Preferably, these are alkali salts of formononetin.

The present invention describes compositions that at least comprise:

Formononetin and at least one active compound selected from groups (2) to (24) below:

Group (2) Strobilurins of the general formula (II)

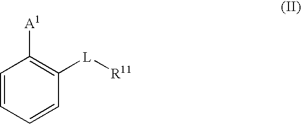

in which
A¹ represents one of the groups

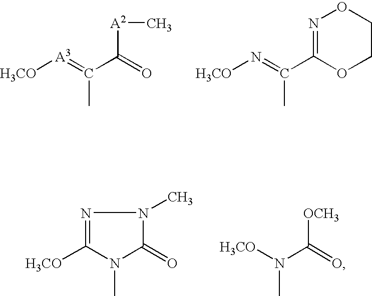

A² represents NH or O,
A³ represents N or CH,
L represents one of the groups

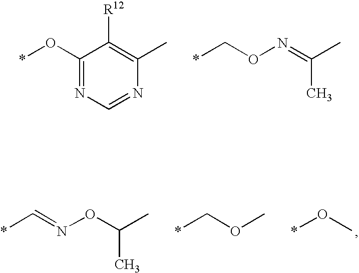

where the bond marked with an asterisk (*) is attached to the phenyl ring,
R¹¹ represents phenyl, phenoxy or pyridinyl, each of which is optionally mono- or disubstituted by identical or different substituents from the group consisting of chlorine, cyano, methyl and trifluoromethyl, or represents 1-(4-chlorophenyl)-pyrazol-3-yl or represents 1,2-propane-dione-bis(O-methyloxime)-1-yl,
R¹² represents hydrogen or fluorine;
   Group (3) Triazoles of the general formula (III)

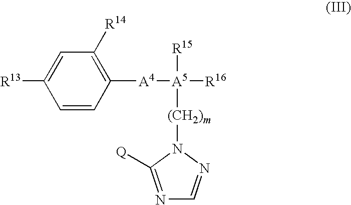

in which
Q represents hydrogen or SH,
m represents 0 or 1,
R¹³ represents hydrogen, fluorine, chlorine, phenyl or 4-chlorophenoxy,
R¹⁴ represents hydrogen or chlorine,
A⁴ represents a direct bond, —CH₂—, —(CH₂)₂— or —O—,
A⁴ furthermore represents *—CH₂—CHR¹⁷— or *—CH═CR¹⁷—, where the bond marked with * is attached to the phenyl ring, in which case R¹⁵ and R¹⁷ together represent —CH₂—CH₂—CH[CH(CH₃)₂]— or —CH₂—CH₂—C(CH₃)₂—,
A⁵ represents C or Si (silicon),
A⁴ further represents —N(R¹⁷)— and A⁵ furthermore together with R¹⁵ and R¹⁶ represents the group C═N—R¹⁸, in which case R¹⁷ and R¹⁸ together represent the group

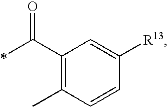

where the bond marked with * is attached to R¹⁷,
R¹⁵ represents hydrogen, hydroxyl or cyano,
R¹⁶ represents 1-cyclopropylethyl, 1-chlorocyclopropyl, C₁-C₄-alkyl, C₁-C₆-hydroxyalkyl, C₁-C₄-alkylcarbonyl, C₁-C₂-haloalkoxy-C₁-C₂-alkyl, trimethylsilyl-C₁-C₂-alkyl, monofluorophenyl or phenyl,
R¹⁵ and R¹⁶ furthermore together represent —O—CH₂—CH(R¹⁸)—O—, —O—CH₂—CH(R¹⁸)—CH₂—, or —O—CH-(2-chlorophenyl)-,
R¹⁸ represents hydrogen, C₁-C₄-alkyl or bromine; or

### Imibenconazole of the Formula

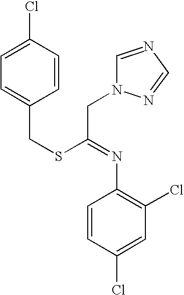

Group (4) Sulphenamides of the general formula (IV)

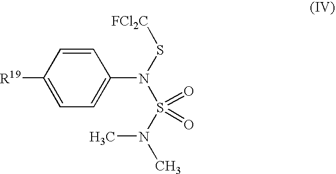

in which R¹⁹ represents hydrogen or methyl;
Group (5) Valinamides selected from
(5-1) iprovalicarb
(5-2) N¹-[2-(4-{[3-(4-chlorophenyl)-2-propynyl]oxy}-3-methoxyphenyl)ethyl]-N²-(methylsulphonyl)-D-valinamide
(5-3) benthiavalicarb
   Group (6) Carboxamides of the general formula (V)

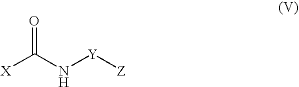

in which
X represents 2-chloro-3-pyridinyl, represents 1-methylpyrazol-4-yl which is substituted in the 3-position by methyl or trifluoromethyl and in the 5-position by hydrogen or chlorine, represents 4-ethyl-2-ethylamino-1,3-thiazol-5-yl, represents 1-methyl-cyclohexyl, represents 2,2-dichloro-1-ethyl-3-methylcyclopropyl, represents 2-fluoro-2-propyl or represents phenyl which is mono- to trisubstituted by identical or different substituents from the group consisting of chlorine, methyl, and trifluoromethyl,
X furthermore represents 3,4-dichloroisothiazole-5-yl, 5,6-dihydro-2-methyl-1,4-oxathiin-3-yl, 4-methyl-1,2,3-thiadiazol-5-yl, 4,5-dimethyl-2-trimethylsilylthiophene-3-yl, 1-methylpyrrol-3-yl which is substituted in the 4-position by methyl or trifluoromethyl and in the 5-position by hydrogen or chlorine,
Y represents a direct bond, C₁-C₆-alkanediyl (alkylene) which is optionally substituted by chlorine, cyano or oxo or represents thiophenediyl,
Y furthermore represents C₂-C₆-alkenediyl (alkenylene),
Z represents hydrogen or the group

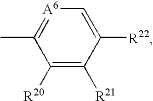

Z furthermore represents C₁-C₆-alkyl,
A⁶ represents CH or N,
R²⁰ represents hydrogen, chlorine, phenyl which is optionally mono- or disubstituted by identical or different substituents from the group consisting of chlorine and di(C₁-C₃-alkyl)amino-carbonyl,
R²⁰ furthermore represents cyano or C₁-C₆-alkyl,
R²¹ represents hydrogen, chlorine, or 1-methylethoxy
R²² represents hydrogen, chlorine, hydroxyl, methyl or trifluoromethyl,
R²² furthermore represents di(C₁-C₃-alkyl)aminocarbonyl,
R²⁰ and R²¹ furthermore together represent *—CH(CH₃)—CH₂—C(CH₃)₂— or *—CH(CH₃)—O—C(CH₃)₂— where the bond marked with * is attached to R²⁰;
   Group (6a) Carboxamides of the general formula (Va)

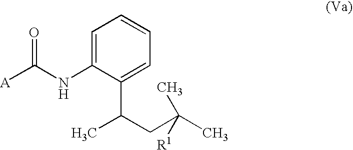

in which
R¹ represents hydrogen, halogen, C₁-C₃-alkyl or C₁-C₃-haloalkyl having 1 to 7 fluorine, chlorine and/or bromine atoms,
A represents one of the radicals A1 to A8 below:

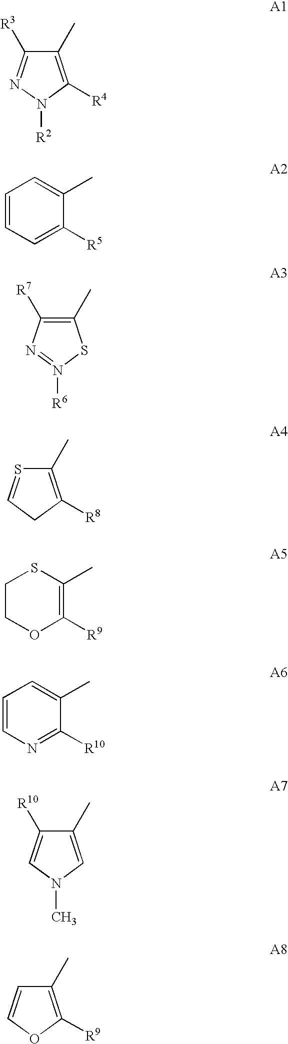

R² represents C₁-C₃-alkyl,
R³ represents hydrogen, halogen, C₁-C₃-alkyl or C₁-C₃-haloalkyl having 1 to 7 fluorine, chlorine and/or bromine atoms,
R⁴ represents hydrogen, halogen or C₁-C₃-alkyl,
R⁵ represents halogen, C₁-C₃-alkyl or C₁-C₃-haloalkyl having 1 to 7 fluorine, chlorine and/or bromine atoms,
R⁶ represents hydrogen, halogen, C₁-C₃-alkyl, amino, mono- or di(C₁-C₃-alkyl)amino,
R⁷ represents hydrogen, halogen, C₁-C₃-alkyl or C₁-C₃-haloalkyl having 1 to 7 fluorine, chlorine and/or bromine atoms,
R⁸ represents halogen, C₁-C₃-alkyl or C₁-C₃-haloalkyl having 1 to 7 fluorine, chlorine and/or bromine atoms,
R⁹ represents halogen, C₁-C₃-alkyl or C₁-C₃-haloalkyl having 1 to 7 fluorine, chlorine and/or bromine atoms,
R¹⁰ represents hydrogen, halogen, C₁-C₃-alkyl or C₁-C₃-haloalkyl having 1 to 7 fluorine, chlorine and/or bromine atoms,

### Group (6b) Carboxamides

(6b-1) Thifluzamide (CII not covered by general formula V)

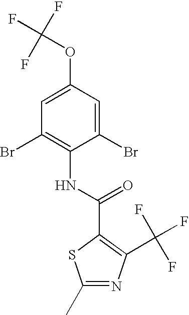

(6b-2) N-(2-[1,1′-bicyclopropyl]-2-ylphenyl)-3-(difluoromethyl)-1-methyl-1H-pyrazole-4-carboxamide

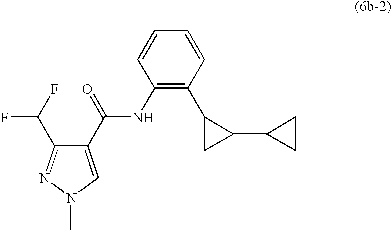

with preference of the following two stereoisomers

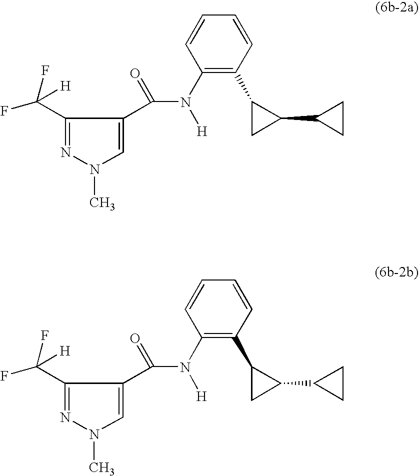

Group (7) Dithiocarbamates selected from
(7-1) mancozeb
(7-2) maneb
(7-3) metiram
(7-4) propineb
(7-5) thiram
(7-6) zineb
(7-7) ziram
   Group (8) Acylalanines of the general formula (VI)

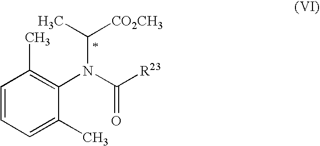

in which
* marks a carbon atom in the R or the S configuration, preferably in the S configuration,
R²³ represents benzyl, furyl or methoxymethyl;
   Group (9): Anilinopyrimidines of the general formula (VII)

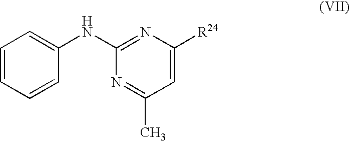

in which
R²⁴ represents methyl, cyclopropyl or 1-propynyl;
   Group (10): Benzimidazoles of the general formula (VIII)

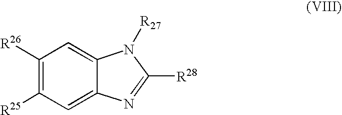

in which
R²⁵ and R²⁶ each represent hydrogen or together represent —O—CF₂—O—,
R²⁷ represents hydrogen, C₁-C₄-alkylaminocarbonyl or represents 3,5-dimethylisoxazol-4-ylsulphonyl,
R²⁸ represents chlorine, methoxycarbonylamino, chlorophenyl, furyl or thiazolyl;
   Group (11): Carbamates of the general formula (IX)

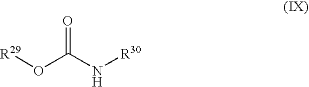

in which
R²⁹ represents n- or isopropyl, methyl.
R³⁰ represents di(C₁-C₂-alkyl)amino-C₂-C₄-alkyl, diethoxyphenyl, [2-Chloro-5-[(1E)-1-[[(6-methyl-2-pyridinyl)methoxy]imino]ethyl]phenyl]methyl or
   salts of these compounds being included;
   Group (12): Dicarboximides selected from
(12-1) captafol
(12-2) captan
(12-3) folpet
(12-4) iprodione
(12-5) procymidone
(12-6) vinclozolin
   Group (13): Guanidines selected from
(13-1) dodine
(13-2) guazatine
(13-3) iminoctadine triacetate
(13-4) iminoctadine tris(albesilate)
   Group (14): Imidazoles selected from
(14-1) cyazofamid
(14-2) prochloraz
(14-3) triazoxide
(14-4) pefurazoate
   Group (15): Morpholines of the general formula (X)

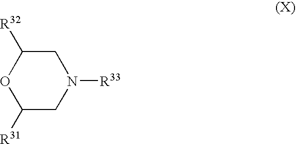

in which
R³¹ and R³² independently of one another represent hydrogen or methyl,
R³³ represents C₁-C₁₄-alkyl (preferably C₁₂-C₁₄-alkyl), C₅-C₁₂-cycloalkyl (preferably C₁₀-C₁₂-cycloalkyl), phenyl-C₁-C₄-alkyl, which may be substituted in the phenyl moiety by halogen or C₁-C₄-alkyl or represents acrylyl which is substituted by chlorophenyl and dimethoxyphenyl;
   Group (16): Pyrroles of the general formula (XI)

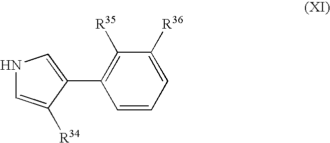

in which
R³⁴ represents chlorine or cyano,
R³⁵ represents chlorine or nitro,
R³⁶ represents chlorine,
R³⁵ and R³⁶ furthermore together represent —O—CF₂—O—;
   Group (17): Phosphonates selected from
(17-1) fosetyl-A1
(17-2) phosphonic acid;
   Group (18): Phenylethanamides of the general formula (XII)

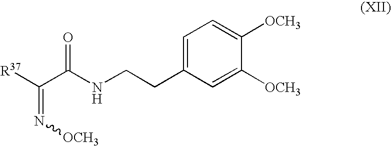

in which
R³⁷ represents unsubstituted or fluorine-, chlorine-, bromine-, methyl- or ethyl-substituted phenyl, 2-naphthyl, 1,2,3,4-tetrahydronaphthyl or indanyl;
   Group (19): Fungicides selected from
(19-1) acibenzolar-S-methyl
(19-2) chlorothalonil
(19-3) cymoxanil
(19-4) edifenphos
(19-5) famoxadone
(19-6) fluazinam
(19-7) copper oxychloride
(19-8) copper hydroxide
(19-9) oxadixyl
(19-10) spiroxamine
(19-11) dithianon
(19-12) metrafenone
(19-13) fenamidone
(19-14) 2,3-dibutyl-6-chlorothieno[2,3-d]pyrimidin-4(3H)-one
(19-15) probenazole
(19-16) isoprothiolane
(19-17) kasugamycin
(19-18) phthalide
(19-19) ferimzone
(19-20) tricyclazole
(19-21) N-({4-[(cyclopropylamino)carbonyl]phenyl}sulphonyl)-2-methoxybenzamide
(19-22) 2-(4-chlorophenyl)-N-{2-[3-methoxy-4-(prop-2-yn-1-yloxy)phenyl]ethyl}-2-(prop-2-yn-1-yloxy)acetamide
(19-23) Diclomezine of the formula

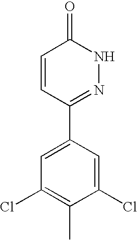

(19-24) Hymexazole of the formula

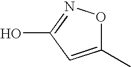

(19-25) Iprobenfos of the formula

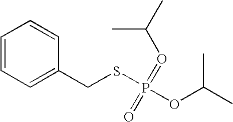

(19-26) Triflumizole of the formula

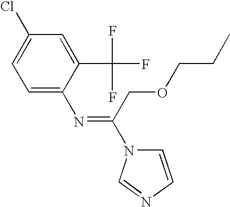

Group (20): (Thio)urea derivatives selected from
(20-1) pencycuron
(20-2) thiophanate-methyl
(20-3) thiophanate-ethyl
   Group (21): Amides of the general formula (XIII)

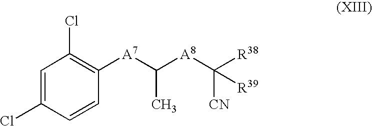

in which
A⁷ represents a direct bond or —O—,
A⁸ represents —C(═O)NH— or —NHC(═O)—,
R³⁸ represents hydrogen or C₁-C₄-alkyl,
R³⁹ represents C₁-C₆-alkyl;
   Group (22): Triazolopyrimidines of the general formula (XIV)

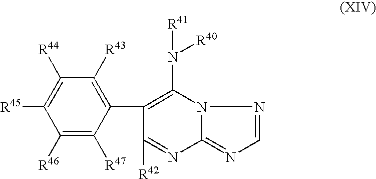

in which
R⁴⁰ represents C₁-C₆-alkyl or C₂-C₆-alkenyl,
R⁴¹ represents C₁-C₆-alkyl,
R⁴⁰ and R⁴¹ furthermore together represent C₄-C₅-alkanediyl (alkylene) which is mono- or disubstituted by C₁-C₆-alkyl,
R⁴² represents bromine or chlorine,
R⁴³ and R⁴⁷ independently of one another represent hydrogen, fluorine, chlorine or methyl,
R⁴⁴ and R⁴⁶ independently of one another represent hydrogen or fluorine,
R⁴⁵ represents hydrogen, fluorine or methyl,
   Group (23): Iodochromones of the general formula (XV)

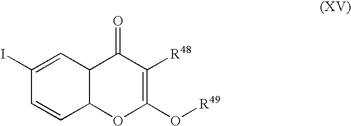

in which
R⁴⁸ represents C₁-C₆-alkyl,
R⁴⁹ represents C₁-C₆-alkyl, C₂-C₆-alkenyl or C₂-C₆-alkynyl;
   Group (24): Biphenylcarboxamides of the general formula (XVI)

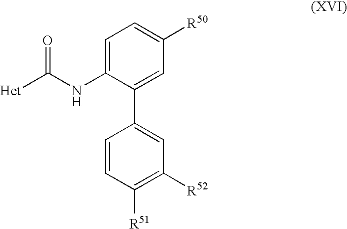

in which
R⁵⁰ represents hydrogen or fluorine,
R⁵¹ represents fluorine, chlorine, bromine, methyl, trifluoromethyl, trifluoromethoxy, —CH═N—OMe or —C(Me)=N—OMe,
R⁵² represents hydrogen, fluorine, chlorine, bromine, methyl or trifluoromethyl,
Het represents one of the radicals Het1 to Het7 below:

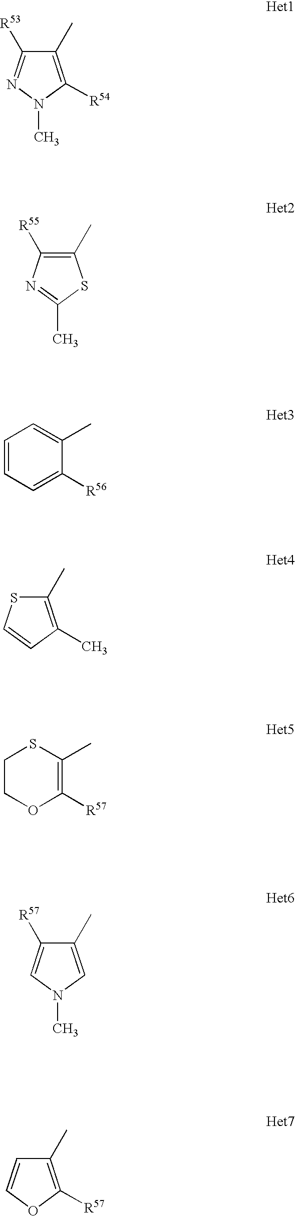

R⁵³ represents iodine, methyl, difluoromethyl or trifluoromethyl,
R⁵⁴ represents hydrogen, fluorine, chlorine or methyl,
R⁵⁵ represents methyl, difluoromethyl or trifluoromethyl,
R⁵⁶ represents chlorine, bromine, iodine, methyl, difluoromethyl or trifluoromethyl,
R⁵⁷ represents methyl or trifluoromethyl.

The formula (II) embraces the following preferred mixing partners of group (2):
(2-1) azoxystrobin (known from EP-A 0 382 375) of the formula

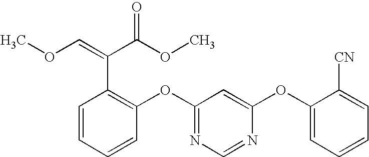

(2-2) fluoxastrobin (known from DE-A 196 02 095) of the formula

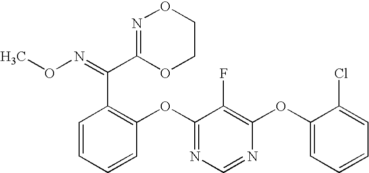

(2-3) (2E)-2-(2-{[6-(3-chloro-2-methylphenoxy)-5-fluoro-4-pyrimidinyl]oxy}phenyl)-2-(methoxy-imino)-N-methylethanamide (known from DE-A 196 46 407, EP-B 0 712 396) of the formula

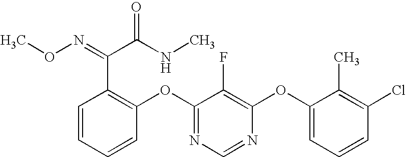

(2-4) trifloxystrobin (known from EP-A 0 460 575) of the formula

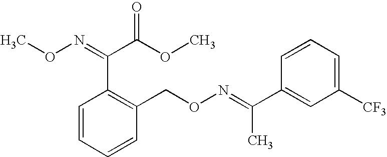

(2-5) (2E)-2-(methoxyimino)-N-methyl-2-(2-{[({(1E)-1-[3-(trifluoromethyl)phenyl]ethyliden}-amino)oxy]methyl}phenyl)ethanamide (known from EP-A 0 569 384) of the formula

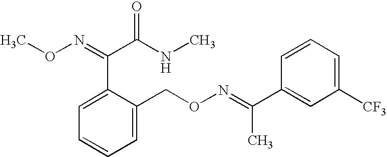

(2-6) (2E)-2-(methoxyimino)-N-methyl-2-{2-[(E)-({1-[3-(trifluoromethyl)phenyl)ethoxy}imino)-methyl]phenyl}ethanamide (known from EP-A 0 596 254) of the formula

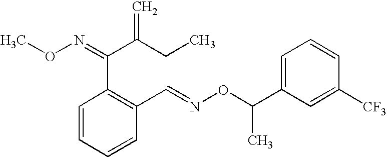

(2-7) orysastrobin (known from DE-A 195 39 324) of the formula

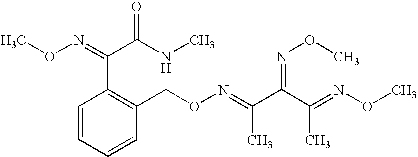

(2-8) 5-methoxy-2-methyl-4-(2-{[({(1E)-1-[3-(trifluoromethyl)phenyl]ethyliden}amino)oxy]-methyl}phenyl)-2,4-dihydro-3H-1,2,4-triazol-3-one (known from WO 98/23155) of the formula

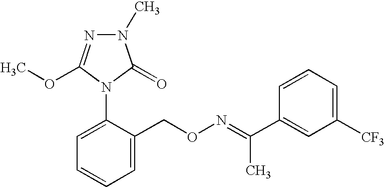

(2-9) kresoxim-methyl (known from EP-A 0 253 213) of the formula

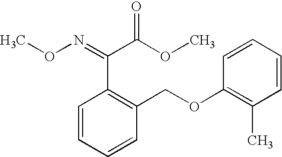

(2-10) dimoxystrobin (known from EP-A 0 398 692) of the formula

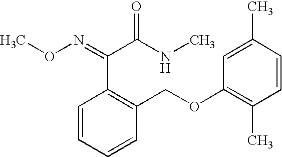

(2-11) picoxystrobin (known from EP-A 0 278 595) of the formula

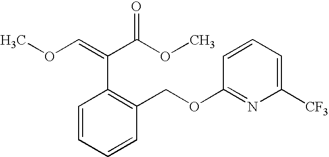

(2-12) pyraclostrobin (known from DE-A 44 23 612) of the formula

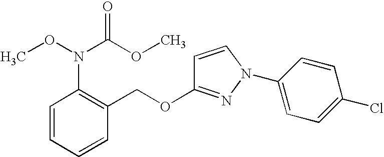

(2-13) metominostrobin (known from EP-A 0 398 692) of the formula

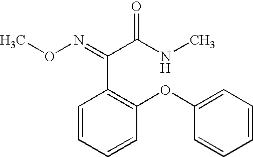

The formula (III) embraces the following preferred mixing partners of group (3):
(3-1) azaconazole (known from DE-A 25 51 560) of the formula

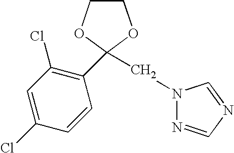

(3-2) etaconazole (known from DE-A 25 51 560) of the formula

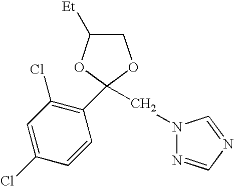

(3-3) propiconazole (known from DE-A 25 51 560) of the formula

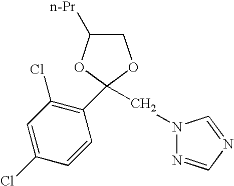

(3-4) difenoconazole (known from EP-A 0 112 284) of the formula

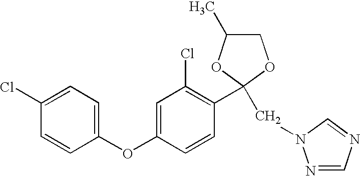

(3-5) bromuconazole (known from EP-A 0 258 161) of the formula

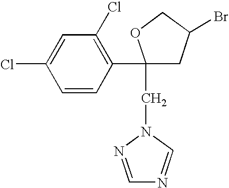

(3-6) cyproconazole (known from DE-A 34 06 993) of the formula

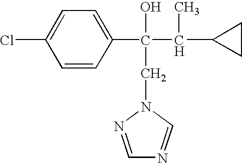

(3-7) hexaconazole (known from DE-A 30 42 303) of the formula

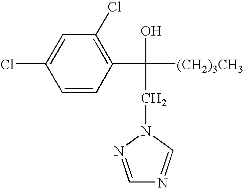

(3-8) penconazole (known from DE-A 27 35 872) of the formula

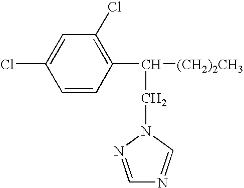

(3-9) myclobutanil (known from EP-A 0 145 294) of the formula

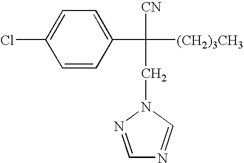

(3-10) tetraconazole (known from EP-A 0 234 242) of the formula

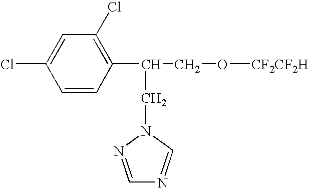

(3-11) flutriafol (known from EP-A 0 015 756) of the formula

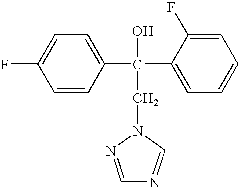

(3-12) epoxiconazole (known from EP-A 0 196 038) of the formula

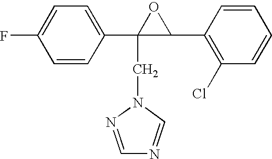

(3-13) flusilazole (known from EP-A 0 068 813) of the formula

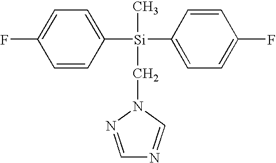

(3-14) simeconazole (known from EP-A 0 537 957) of the formula

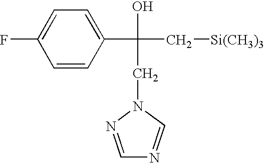

(3-15) prothioconazole (known from WO 96/16048) of the formula

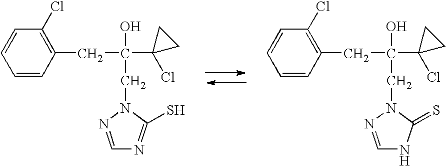

(3-16) fenbuconazole (known from DE-A 37 21 786) of the formula

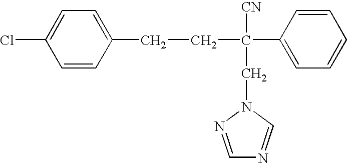

(3-17) tebuconazole (known from EP-A 0 040 345) of the formula

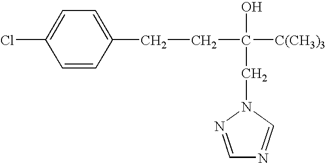

(3-18) ipconazole (known from EP-A 0 329 397) of the formula

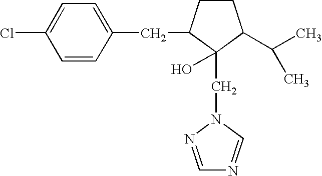

(3-19) metconazole (known from EP-A 0 329 397) of the formula

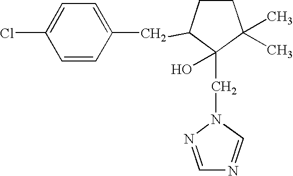

(3-20) triticonazole (known from EP-A 0 378 953) of the formula

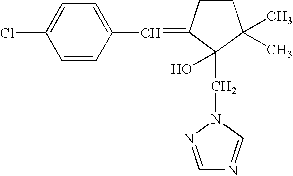

(3-21) bitertanol (known from DE-A 23 24 010) of the formula

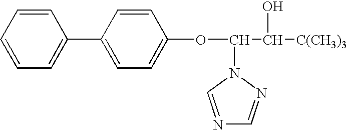

(3-22) triadimenol (known from DE-A 23 24 010) of the formula

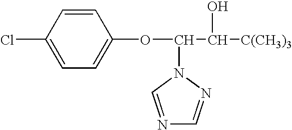

(3-23) triadimefon (known from DE-A 22 01 063) of the formula

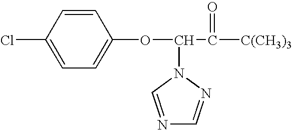

(3-24) fluquinconazole (known from EP-A 0 183 458) of the formula

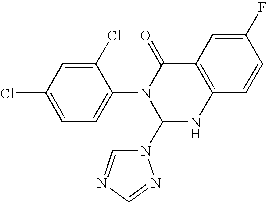

(3-25) quinconazole (known from EP-A 0 183 458) of the formula

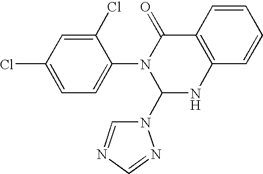

(3-26) Diclobutrazole of the formula

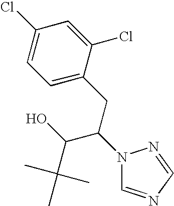

(3-27) Diniconazole of the formula

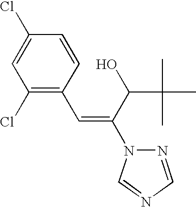

The formula (IV) embraces the following preferred mixing partners of group (4):
(4-1) dichlofluanid (known from DE-A 11 93 498) of the formula

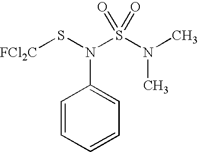

(4-2) tolylfluanid (known from DE-A 11 93 498) of the formula

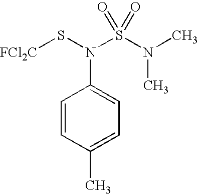

Preferred mixing partners of group (5) are
(5-1) iprovalicarb (known from DE-A 40 26 966) of the formula

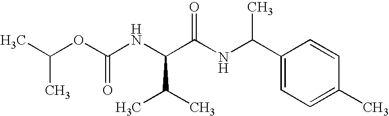

(5-3) benthiavalicarb (known from WO 96/04252) of the formula

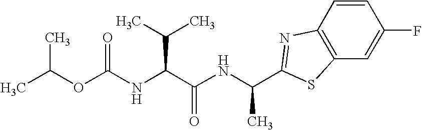

The formula (V) embraces the following preferred mixing partners of group (6):
(6-1) 2-chloro-N-(1,1,3-trimethylindan-4-yl)nicotinamide (known from EP-A 0 256 503) of the formula

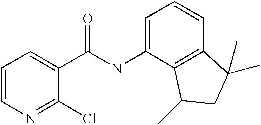

(6-2) boscalid (known from DE-A 195 31 813) of the formula

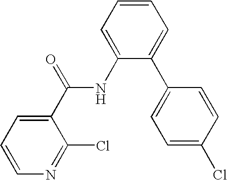

(6-3) furametpyr (known from EP-A 0 315 502) of the formula

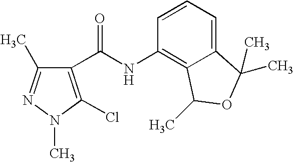

(6-4) N-(3-p-tolylthiophen-2-yl)-1-methyl-3-trifluoromethyl-1H-pyrazole-4-carboxamide (known from EP-A 0 737 682) of the formula

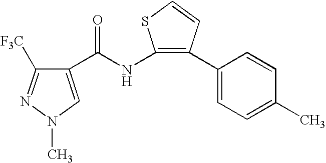

(6-5) ethaboxam (known from EP-A 0 639 574) of the formula

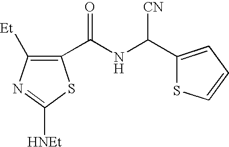

(6-6) fenhexamid (known from EP-A 0 339 418) of the formula

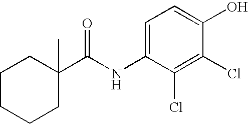

(6-7) carpropamid (known from EP-A 0 341 475) of the formula

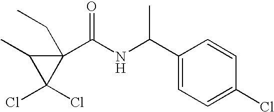

(6-8) 2-chloro-4-(2-fluoro-2-methylpropionylamino)-N,N-dimethylbenzamide (known from EP-A 0 600 629) of the formula

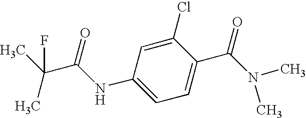

(6-9) picobenzamid (known from WO 99/42447) of the formula

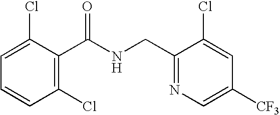

(6-10) zoxamide (known from EP-A 0 604 019) of the formula

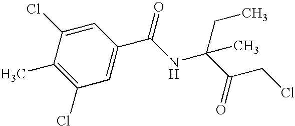

(6-11) 3,4-dichloro-N-(2-cyanophenyl)isothiazole-5-carboxamide (Isothianil) (known from WO 99/24413) of the formula

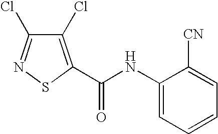

(6-12) carboxin (known from U.S. Pat. No. 3,249,499) of the formula

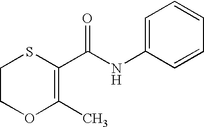

(6-13) tiadinil (known from U.S. Pat. No. 6,616,054) of the formula

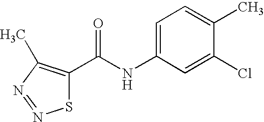

(6-14) penthiopyrad (known from EP-A 0 737 682) of the formula

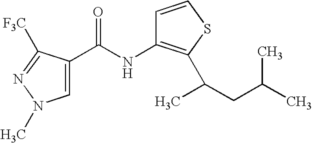

(6-15) silthiofam (known from WO 96/18631) of the formula

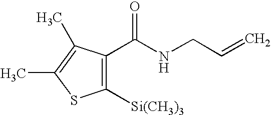

(6-16) N-[2-(1,3-dimethylbutyl)phenyl]-1-methyl-4-(trifluoromethyl)-1H-pyrrole-3-carboxamide (known from WO 02/38542) of the formula

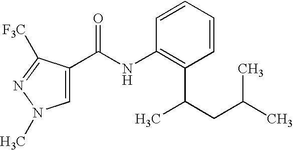

(6-17) N-{2-[3-chloro-5-(trifluoromethyl)pyridin-2-yl]ethyl}-2-(trifluoromethyl)benzamide (known from WO04016088)

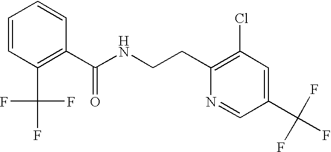

(6-18) flutolanil of the formula

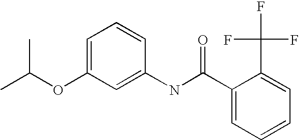

(6b-2) N-(2-[1,1′-bicyclopropyl]-2-ylphenyl)-3-(difluoromethyl)-1-methyl-1H-pyrazole-4-carboxamide

The formula (Va) embraces the following preferred mixing partners of group (6a):
R¹ represents hydrogen, fluorine, chlorine, methyl, ethyl, n-, isopropyl, monofluoromethyl, difluoromethyl, trifluoromethyl, monochloromethyl, dichloromethyl or trichloromethyl,
A represents one of the radicals A1 to A5 below:

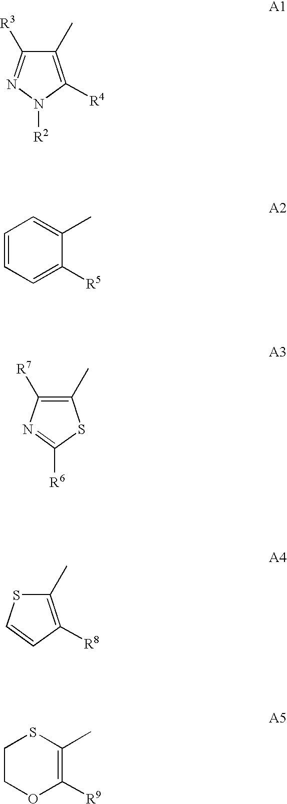

R² represents methyl, ethyl, n- or isopropyl,
R³ represents iodine, methyl, difluoromethyl or trifluoromethyl,
R⁴ represents hydrogen, fluorine, chlorine or methyl,
R⁵ represents chlorine, bromine, iodine, methyl, difluoromethyl or trifluoromethyl,
R⁶ represents hydrogen, chlorine, methyl, amino or dimethylamino,
R⁷ represents methyl, difluoromethyl or trifluoromethyl,
R⁸ represents bromine or methyl,
R⁹ represents methyl or trifluoromethyl.

Particular preference is given to carboxamides of the formula (Va) in which
R¹ represents hydrogen, fluorine, chlorine, methyl, ethyl or trifluoromethyl,
A represents one of the radicals A1 or A2 below:

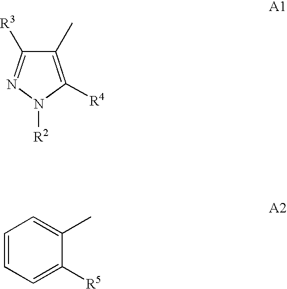

R² represents methyl or isopropyl,
R³ represents methyl, difluoromethyl or trifluoromethyl,
R⁴ represents hydrogen or fluorine,
R⁵ represents iodine, difluoromethyl or trifluoromethyl.

Very particular preference is given to carboxamides of the formula (Va) in which
R¹ represents hydrogen or methyl,
A represents one of the radicals A1 or A2 below:

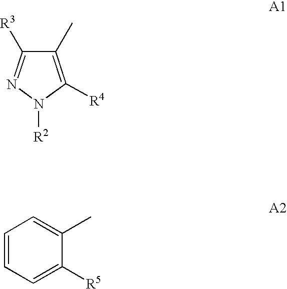

R² represents methyl,
R³ represents methyl,
R⁴ represents fluorine,
R⁵ represents iodine or trifluoromethyl.

Very particular preference is given to using, in mixtures, compounds of the formula (Va)

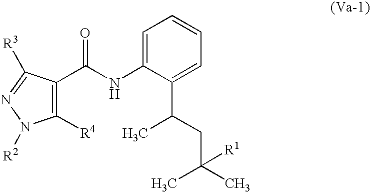

in which R¹, R², R³ and R⁴ are as defined above.

Very particular preference is given to using, in mixtures, compounds of the formula (Vb)

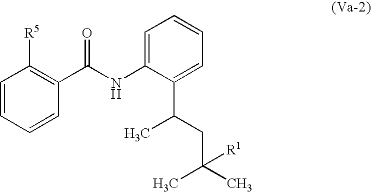

in which R¹ and R⁵ are as defined above.

The formula (Va) embraces in particular the following preferred mixing partners of group (6a):
(Va1-1) N-[2-(1,3-dimethylbutyl)phenyl]-1,3-dimethyl-1H-pyrazole-4-carboxamide
(6a-2) N-[2-(1,3-dimethylbutyl)phenyl]-5-fluoro-1,3-dimethyl-1H-pyrazole-4-carboxamide (known from WO 03/010149)
(6a-3) N-[2-(1,3-dimethylbutyl)phenyl]-5-chloro-1,3-dimethyl-1H-pyrazole-4-carboxamide (known from JP-A 10-251240)
(6a-4) 3-(difluoromethyl)-N-[2-(1,3-dimethylbutyl)phenyl]-1-methyl-1H-pyrazole-4-carboxamide
(6a-5) 3-(trifluoromethyl)-N-[2-(1,3-dimethylbutyl)phenyl]-5-fluoro-1-methyl-1H-pyrazole-4-carboxamide (known from DE-A 103 03 589)
(6a-6) 3-(trifluoromethyl)-N-[2-(1,3-dimethylbutyl)phenyl]-5-chloro-1-methyl-1H-pyrazole-4-carboxamide (known from JP-A 10-251240)
(6a-7) 1,3-dimethyl-N-[2-(1,3,3-trimethylbutyl)phenyl]-1H-pyrazole-4-carboxamide (known from JP-A 10-251240)
(6a-8) 5-fluoro-1,3-dimethyl-N-[2-(1,3,3-trimethylbutyl)phenyl]-1H-pyrazole-4-carboxamide (known from WO 03/010149)
(6a-9) 3-(difluoromethyl)-1-methyl-N-[2-(1,3,3-trimethylbutyl)phenyl]-1H-pyrazole-4-carboxamide
(6a-10) 3-(trifluoromethyl)-1-methyl-N-[2-(1,3,3-trimethylbutyl)phenyl]-1H-pyrazole-4-carboxamide
(6a-11) 3-(trifluoromethyl)-5-fluoro-1-methyl-N-[2-(1,3,3-trimethylbutyl)phenyl]-1H-pyrazole-4-carboxamide (known from DE-A 103 03 589)
(6a-12) 3-(trifluoromethyl)-5-chloro-1-methyl-N-[2-(1,3,3-trimethylbutyl)phenyl]-1H-pyrazole-4-carboxamide (known from JP-A 10-251240)

The formula (Vb) embraces in particular the following preferred mixing partners of group (6a):
(6a-13) N-[2-(1,3-dimethylbutyl)phenyl]-2-iodobenzamide (known from DE-A 102 29 595)
(6a-14) 2-iodo-N-[2-(1,3,3-trimethylbutyl)phenyl]benzamide (known from DE-A 102 29 595)
(6a-15) N-[2-(1,3-dimethylbutyl)phenyl]-2-(trifluoromethyl)benzamide (known from DE-A 102 29 595)
(6a-16) 2-(trifluoromethyl)-N-[2-(1,3,3-trimethylbutyl)phenyl]benzamide (known from DE-A 102 29 595)

Preferred mixing partners of group (7) are
(7-1) mancozeb (known from DE-A 12 34 704) having the IUPAC name manganese ethylenebis(dithiocarbamate) (polymeric) complex with zinc salt
(7-2) maneb (known from U.S. Pat. No. 2,504,404) of the formula

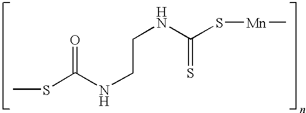

(7-3) metiram (known from DE-A 10 76 434) having the IUPAC name zinc ammoniate ethylenebis(dithiocarbamate)-poly(ethylenethiuram disulphide)
(7-4) propineb (known from GB 935 981) of the formula

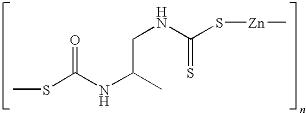

(7-5) thiram (known from U.S. Pat. No. 1,972,961) of the formula

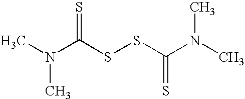

(7-6) zineb (known from DE-A 10 81 446) of the formula

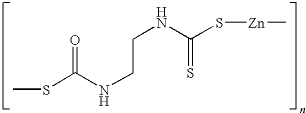

(7-7) ziram (known from U.S. Pat. No. 2,588,428) of the formula

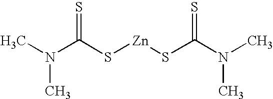

The formula (VI) embraces the following preferred mixing partners of group (8):
(8-1) benalaxyl (known from DE-A 29 03 612) of the formula

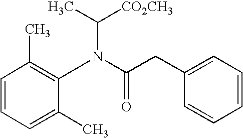

(8-2) furalaxyl (known from DE-A 25 13 732) of the formula

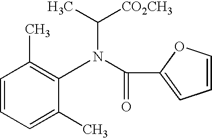

(8-3) metalaxyl (known from DE-A 25 15 091) of the formula

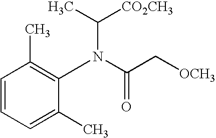

(8-4) metalaxyl-M (known from WO 96/01559) of the formula

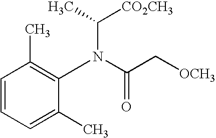

(8-5) benalaxyl-M of the formula

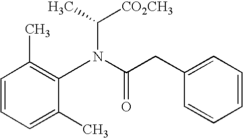

The formula (VII) embraces the following preferred mixing partners of group (9):
(9-1) cyprodinil (known from EP-A 0 310 550) of the formula

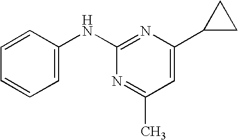

(9-2) mepanipyrim (known from EP-A 0 270 111) of the formula

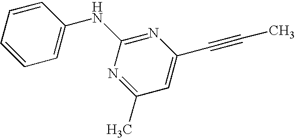

(9-3) pyrimethanil (known from DD 151 404) of the formula

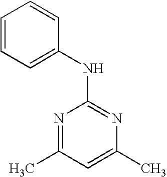

The formula (VIII) embraces the following preferred mixing partners of group (10):
(10-1) 6-chloro-5-[(3,5-dimethylisoxazol-4-yl)sulphonyl]-2,2-difluoro-5H-[1,3]dioxolo[4,5-f]-benzimidazole (known from WO 97/06171) of the formula

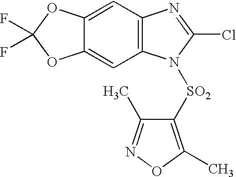

(10-2) benomyl (known from U.S. Pat. No. 3,631,176) of the formula

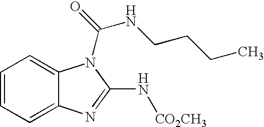

(10-3) carbendazim (known from U.S. Pat. No. 3,010,968) of the formula

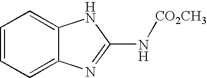

(10-4) chlorfenazole of the formula

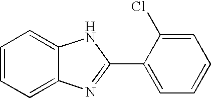

(10-5) fuberidazole (known from DE-A 12 09 799) of the formula

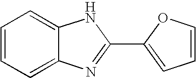

(10-6) thiabendazole (known from U.S. Pat. No. 3,206,468) of the formula

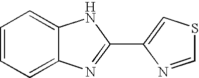

The formula (IX) embraces the following preferred mixing partners of group (11):
(11-1) diethofencarb (known from EP-A 0 078 663) of the formula

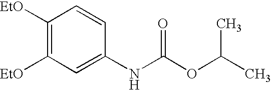

(11-2) propamocarb (known from U.S. Pat. No. 3,513,241) of the formula

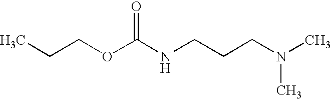

(11-3) propamocarb-hydrochloride (known from U.S. Pat. No. 3,513,241) of the formula

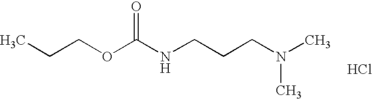

(11-4) propamocarb-fosetyl of the formula

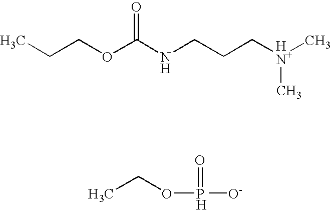

(11-5) pyribencarb of the formula

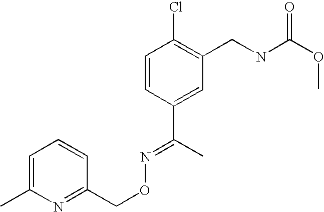

Preferred mixing partners of group (12) are
(12-1) captafol (known from U.S. Pat. No. 3,178,447) of the formula

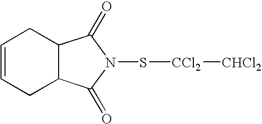

(12-2) captan (known from U.S. Pat. No. 2,553,770) of the formula

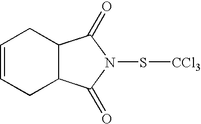

(12-3) folpet (known from U.S. Pat. No. 2,553,770) of the formula

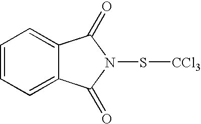

(12-4) iprodione (known from DE-A 21 49 923) of the formula

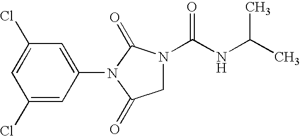

(12-5) procymidone (known from DE-A 20 12 656) of the formula

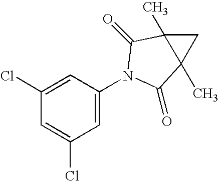

(12-6) vinclozolin (known from DE-A 22 07 576) of the formula

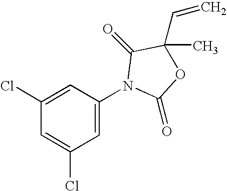

Preferred mixing partners of group (13) are
(13-1) dodine (known from GB 11 03 989) of the formula

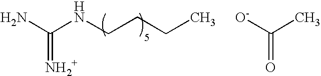

(13-2) guazatine (known from GB 11 14 155)
(13-3) iminoctadine triacetate (known from EP-A 0 155 509) of the formula

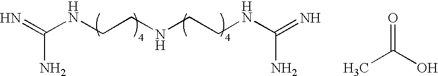

Preferred mixing partners of group (14) are
(14-1) cyazofamid (known from EP-A 0 298 196) of the formula

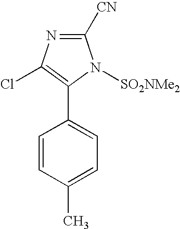

(14-2) prochloraz (known from DE-A 24 29 523) of the formula

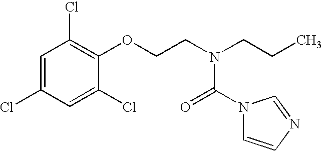

(14-3) triazoxide (known from DE-A 28 02 488) of the formula

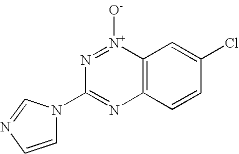

(14-4) pefurazoate (known from EP-A 0 248 086) of the formula

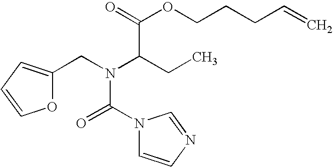

The formula (X) embraces the following preferred mixing partners of group (15):
(15-1) aldimorph (known from DD 140 041) of the formula

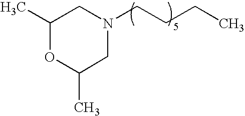

(15-2) tridemorph (known from GB 988 630) of the formula

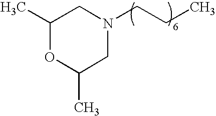

(15-3) dodemorph (known from DE-A 25 432 79) of the formula

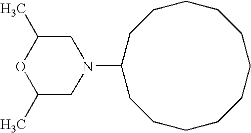

(15-4) fenpropimorph (known from DE-A 26 56 747) of the formula

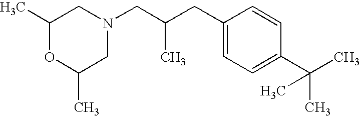

(15-5) dimethomorph (known from EP-A 0 219 756) of the formula

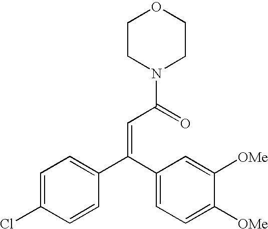

The formula (XI) embraces the following preferred mixing partners of group (16):
(16-1) fenpiclonil (known from EP-A 0 236 272) of the formula

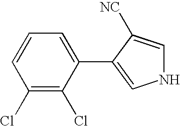

(16-2) fludioxonil (known from EP-A 0 206 999) of the formula

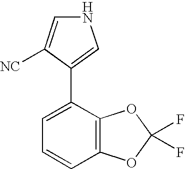

(16-3) pyrrolnitrin (known from JP 65-25876) of the formula

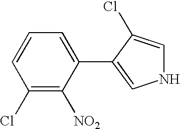

Preferred mixing partners of group (17) are
(17-1) fosetyl-A1 (known from DE-A 24 56 627) of the formula

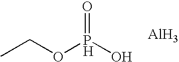

(17-2) phosphonic acid (known chemical) of the formula

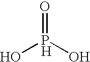

The formula (XII) embraces the following preferred mixing partners of group (18) which are known from WO 96/23793 and can in each case be present as E or Z isomers. Accordingly, compounds of the formula (XII) can be present as a mixture of different isomers or else in the form of a single isomer. Preference is given to compounds of the formula (XII) in the form of their E isomers:
(18-1) the compound 2-(2,3-dihydro-1H-inden-5-yl)-N-[2-(3,4-dimethoxyphenyl)ethyl]-2-(methoxyimino)acetamide of the formula

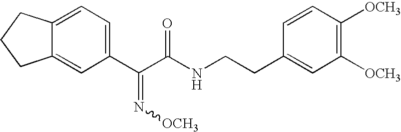

(18-2) the compound N-[2-(3,4-dimethoxyphenyl)ethyl]-2-(methoxyimino)-2-(5,6,7,8-tetrahydro-naphthalen-2-yl)acetamide of the formula

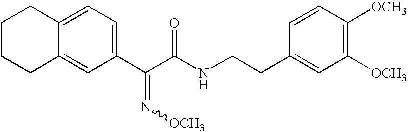

(18-3) the compound 2-(4-chlorophenyl)-N-[2-(3,4-dimethoxyphenyl)ethyl]-2-(methoxyimino)-acetamide of the formula

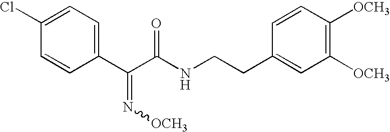

(18-4) the compound 2-(4-bromophenyl)-N-[2-(3,4-dimethoxyphenyl)ethyl]-2-(methoxyimino)-acetamide of the formula

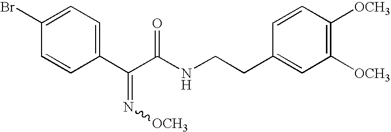

(18-5) the compound 2-(4-methylphenyl)-N-[2-(3,4-dimethoxyphenyl)ethyl]-2-(methoxyimino)-acetamide of the formula

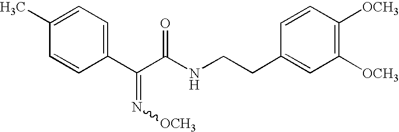

(18-6) the compound 2-(4-ethylphenyl)-N-[2-(3,4-dimethoxyphenyl)ethyl]-2-(methoxyimino)-acetamide of the formula

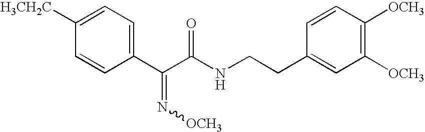

Preferred mixing partners of group (19) are (19-1) acibenzolar-S-methyl (known from EP-A 0 313 512) of the formula

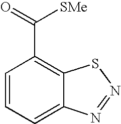

(19-2) chlorothalonil (known from U.S. Pat. No. 3,290,353) of the formula

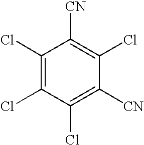

(19-3) cymoxanil (known from DE-A 23 12 956) of the formula

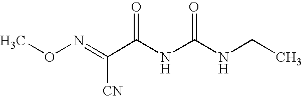

(19-4) edifenphos (known from DE-A 14 93 736) of the formula

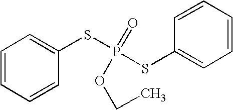

(19-5) famoxadone (known from EP-A 0 393 911) of the formula

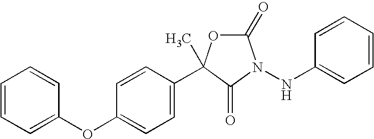

(19-6) fluazinam (known from EP-A 0 031 257) of the formula

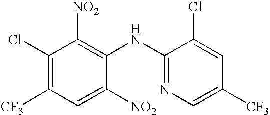

(19-7) copper oxychloride
(19-9) oxadixyl (known from DE-A 30 30 026) of the formula

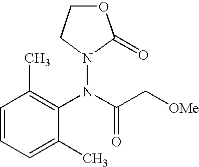

(19-10) spiroxamine (known from DE-A 37 35 555) of the formula

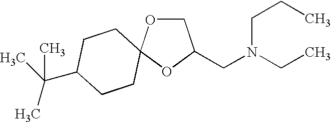

(19-11) dithianon (known from JP-A 44-29464) of the formula

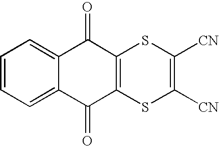

(19-12) metrafenone (known from EP-A 0 897 904) of the formula

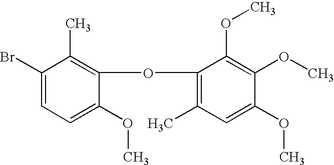

(19-13) fenamidone (known from EP-A 0 629 616) of the formula

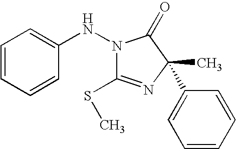

(19-14) 2,3-dibutyl-6-chlorothieno[2,3-d]pyrimidin-4(3H)one (known from WO 99/14202) of the formula

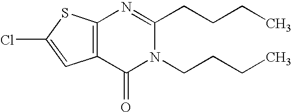

(19-15) probenazole (known from U.S. Pat. No. 3,629,428) of the formula

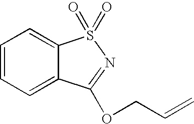

(19-16) isoprothiolane (known from U.S. Pat. No. 3,856,814) of the formula

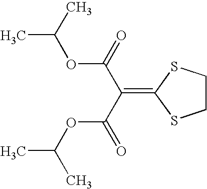

(19-17) kasugamycin (known from GB 1 094 567) of the formula

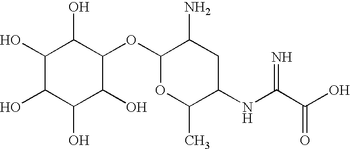

(19-18) phthalide (known from JP-A 57-55844) of the formula

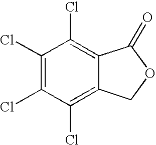

(19-19) ferimzone (known from EP-A 0 019 450) of the formula

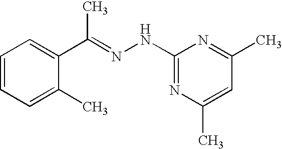

(19-20) tricyclazole (known from DE-A 22 50 077) of the formula

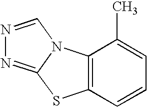

(19-21) N-({4-[(cyclopropylamino)carbonyl]phenyl}sulphonyl)-2-methoxybenzamide of the formula

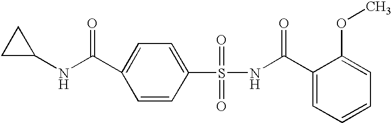

(19-22) 2-(4-chlorophenyl)-N-{2-[3-methoxy-4-(prop-2-yn-1-yloxy)phenyl]ethyl}-2-(prop-2-yn-1-yloxy)acetamide (known from WO 01/87822) of the formula

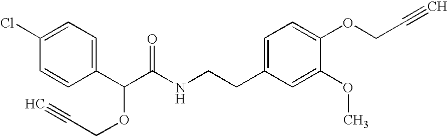

Preferred mixing partners of group (20) are (20-1) pencycuron (known from DE-A 27 32 257) of the formula

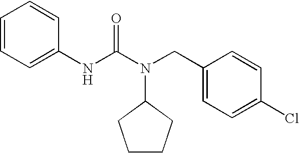

(20-2) thiophanate-methyl (known from DE-A 18 06 123) of the formula

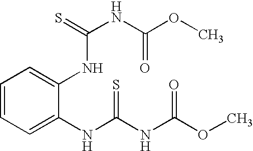

(20-3) thiophanate-ethyl (known from DE-A 18 06 123) of the formula

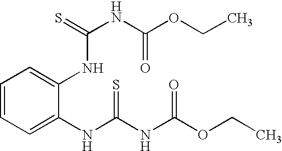

Preferred mixing partners of group (21) are (21-1) fenoxanil (known from EP-A 0 262 393) of the formula

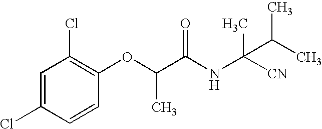

(21-2) diclocymet (known from JP-A 7-206608) of the formula

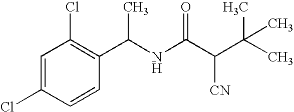

Preferred mixing partners of group (22) are
(22-1) 5-chloro-N-[(1S)-2,2,2-trifluoro-1-methylethyl]-6-(2,4,6-trifluorophenyl)[1,2,4]triazolo-[1,5-a]pyrimidine-7-amine (known from U.S. Pat. No. 5,986,135) of the formula

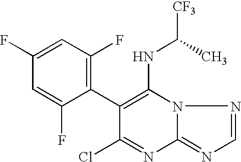

(22-2) 5-chloro-N-[(1R)-1,2-dimethylpropyl]-6-(2,4,6-trifluorophenyl)[1,2,4]triazolo[1,5-a]-pyrimidine-7-amine (known from WO 02/38565) of the formula

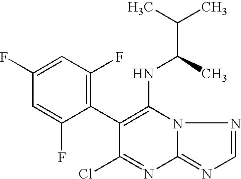

(22-3) 5-chloro-6-(2-chloro-6-fluorophenyl)-7-(4-methylpiperidin-1-yl)[1,2,4]triazolo[1,5-a]-pyrimidine (known from U.S. Pat. No. 5,593,996) of the formula

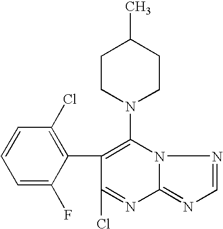

(22-4) 5-chloro-6-(2,4,6-trifluorophenyl)-7-(4-methylpiperidin-1-yl)[1,2,4]triazolo[1,5-a]pyrimidine (known from DE-A 101 24 208) of the formula

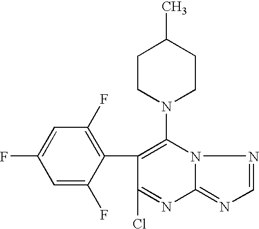

Preferred mixing partners of group (23) are
(23-1) 2-butoxy-6-iodo-3-propylbenzopyran-4-one (known from WO 03/014103) of the formula

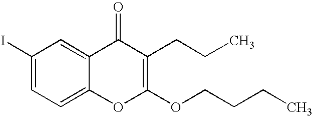

(23-2) 2-ethoxy-6-iodo-3-propylbenzopyran-4-one (known from WO 03/014103) of the formula

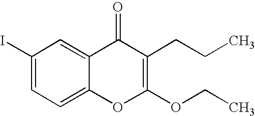

(23-3) 6-iodo-2-propoxy-3-propylbenzopyran-4-one (known from WO 03/014103) of the formula

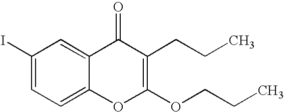

(23-4) 2-but-2-ynyloxy-6-iodo-3-propylbenzopyran-4-one (known from WO 03/014103) of the formula

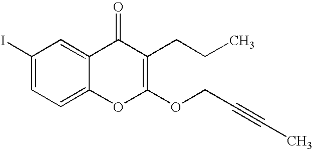

(23-5) 6-iodo-2-(1-methylbutoxy)-3-propylbenzopyran-4-one (known from WO 03/014103) of the formula

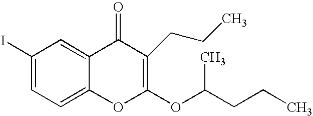

(23-6) 2-but-3-enyloxy-6-iodobenzopyran-4-one (known from WO 03/014103) of the formula

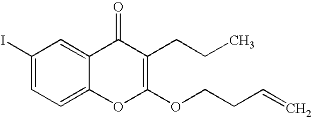

(23-7) 3-butyl-6-iodo-2-isopropoxybenzopyran-4-one (known from WO 03/014103) of the formula

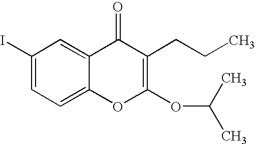

Preferred mixing partners of group (24) are
(24-1) N-(3′,4′-dichloro-5-fluoro-1,1′-biphenyl-2-yl)-3-(difluoromethyl)-1-methyl-1H-pyrazole-4-carboxamide (known from WO 03/070705) of the formula

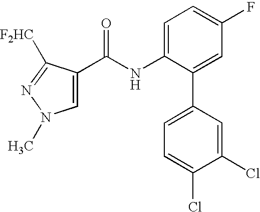

(24-2) 3-(difluoromethyl)-N-{3′-fluoro-4′-[(E)-(methoxyimino)methyl]-1,1′-biphenyl-2-yl}-1-methyl-1H-pyrazole-4-carboxamide (known from WO 02/08197) of the formula

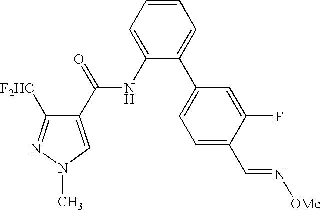

(24-3) 3-(trifluoromethyl)-N-{3′-fluoro-4′-[(E)-(methoxyimino)methyl]-1,1′-biphenyl-2-yl}-1-methyl-1H-pyrazole-4-carboxamide (known from WO 02/08197) of the formula

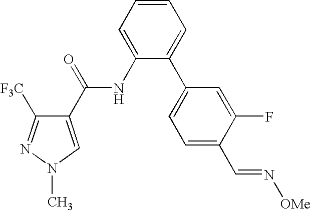

(24-4) N-(3′,4′-dichloro-1,1′-biphenyl-2-yl)-5-fluoro-1,3-dimethyl-1H-pyrazole-4-carboxamide (known from WO 00/14701) of the formula

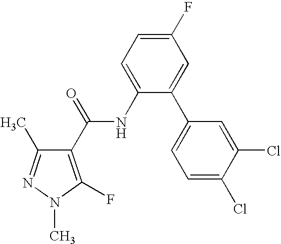

(24-5) N-(4′-chloro-3′-fluoro-1,1′-biphenyl-2-yl)-2-methyl-4-(trifluoromethyl)-1,3-thiazole-5-carboxamide (known from WO 03/066609) of the formula

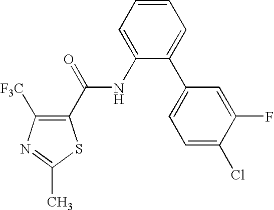

(24-6) N-(4′-chloro-1,1′-biphenyl-2-yl)-4-(difluoromethyl)-2-methyl-1,3-thiazole-5-carboxamide (known from WO 03/066610) of the formula

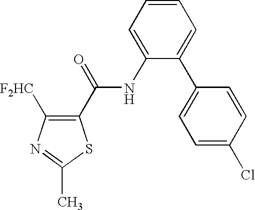

(24-7) N-(4′-bromo-1,1′-biphenyl-2-yl)-4-(difluoromethyl)-2-methyl-1,3-thiazole-5-carboxamide (known from WO 03/066610) of the formula

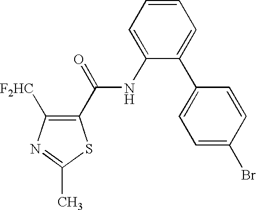

(24-8) 4-(difluoromethyl)-2-methyl-N-[4′-(trifluoromethyl)-1,1′-biphenyl-2-yl]-1,3-thiazole-5-carboxamide (known from WO 03/066610) of the formula

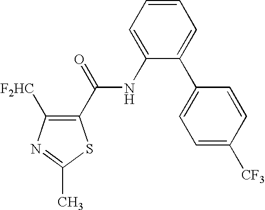

Compound (6-7), carpropamid, has three asymmetrically substituted carbon atoms. Accordingly, compound (6-7) can be present as a mixture of different isomers or else in the form of a single component. Particular preference is given to the compounds
(1S,3R)-2,2-dichloro-N-[(1R)-1-(4-chlorophenyl)ethyl]-1-ethyl-3-methylcyclopropanecarboxamide of the formula

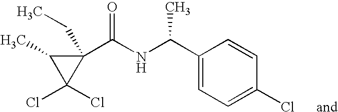

(1R,3S)-2,2-dichloro-N-[(1R)-1-(4-chlorophenyl)ethyl]-1-ethyl-3-methylcyclopropanecarboxamide of the formula

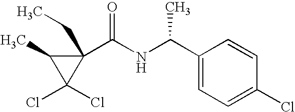

Particularly preferred mixing partners are the following active compounds:
(2-1) azoxystrobin
(2-2) fluoxastrobin
(2-3) (2E)-2-(2-{[6-(3-chloro-2-methylphenoxy)-5-fluoro-4-pyrimidinyl]oxy}phenyl)-2-(methoxyimino)-N-methylethanamide
(2-4) trifloxystrobin
(2-5) (2E)-2-(methoxyimino)-N-methyl-2-(2-{[{(1E)-1-[3-(trifluoromethyl)-phenyl]ethyliden}amino)oxy]methyl}phenyl)ethanamide
(2-6) (2E)-2-(methoxyimino)-N-methyl-2-{2-[(E)-({1-[3-(trifluoromethyl)phenyl]-ethoxy}imino)methyl]phenyl}ethanamide
(2-8) 5-methoxy-2-methyl-4-(2-{[({(1E)-1-[3-(trifluoromethyl)phenyl]ethyliden}-amino)oxy]methyl}phenyl)-2,4-dihydro-3H-1,2,4-triazol-3-one
(2-11) picoxystrobin
(2-9) kresoxim-methyl
(2-10) dimoxystrobin
(2-12) pyraclostrobin
(2-13) metominostrobin
(3-3) propiconazole
(3-4) difenoconazole
(3-6) cyproconazole
(3-7) hexaconazole
(3-8) penconazole
(3-9) myclobutanil
(3-10) tetraconazole
(3-12) epoxiconazole
(3-13) flusilazole
(3-15) prothioconazole
(3-16) fenbuconazole
(3-17) tebuconazole
(3-19) metconazole
(3-21) bitertanol
(3-22) triadimenol
(3-23) triadimefon
(3-24) fluquinconazole
(4-1) dichlofluanid
(4-2) tolylfluanid
(5-1) iprovalicarb
(5-3) benthiavalicarb
(6-2) boscalid
(6-5) ethaboxam
(6-6) fenhexamid
(6-7) carpropamid
(6-8) 2-chloro-4-[(2-fluoro-2-methylpropanoyDamino]-N,N-dimethylbenzamide
(6-9) picobenzamid
(6-10) zoxamide
(6-11) 3,4-dichloro-N-(2-cyanophenyl)isothiazole-5-carboxamide
(6-14) penthiopyrad
(6-16) N-[2-(1,3-dimethylbutyl)phenyl]-1-methyl-4-(trifluoromethyl)-1H-pyrrole-3-carboxamide
(6-17) N-{2-[3-chloro-5-(trifluoromethyl)pyridin-2-yl]ethyl}-2-(trifluoromethyl)benzamide
(6b-2) N-(2-[1,1′-bicyclopropyl]-2-ylphenyl)-3-(difluoromethyl)-1-methyl-1H-pyrazole-4-carboxamide
(7-1) mancozeb
(7-2) maneb
(7-4) propineb
(7-5) thiram
(7-6) zineb
(8-1) benalaxyl
(8-2) furalaxyl
(8-3) metalaxyl
(8-4) metalaxyl-M
(8-5) benalaxyl-M
(9-1) cyprodinil
(9-2) mepanipyrim
(9-3) pyrimethanil
(10-1) 6-chloro-5-[(3,5-dimethylisoxazol-4-yl)sulphonyl]-2,2-difluoro-5H-[1,3]dioxolo[4,5-f]-benzimidazole
(10-3) carbendazim
(11-1) diethofencarb
(11-2) propamocarb
(11-3) propamocarb-hydrochloride
(11-4) propamocarb-fosetyl
(12-2) captan
(12-3) folpet
(12-4) iprodione
(12-5) procymidone
(13-1) dodine
(13-2) guazatine
(13-3) iminoctadine triacetate
(14-1) cyazofamid
(14-2) prochloraz
(14-3) triazoxide
(15-5) dimethomorph
(15-4) fenpropimorph
(16-2) fludioxonil
(17-1) fosetyl-A1
(17-2) phosphonic acid
(19-1) acibenzolar-S-methyl
(19-2) chlorothalonil
(19-3) cymoxanil
(19-5) famoxadone
(19-6) fluazinam
(19-9) oxadixyl
(19-10) spiroxamine
(19-7) copper oxychloride
(19-13) fenamidone
(19-22) 2-(4-chlorophenyl)-N-{2-[3-methoxy-4-(prop-2-yn-1-yloxy)phenyl]ethyl}-2-(prop-2-yn-1-yloxy)acetamide
(20-1) pencycuron
(20-2) thiophanate-methyl
(22-1) 5-chloro-N-[(1S)-2,2,2-trifluoro-1-methylethyl]-6-(2,4,6-trifluorophenyl)[1,2,4]-triazolo[1,5-a]pyrimidine-7-amine
(22-2) 5-chloro-N-[(1R)-1,2-dimethylpropyl]-6-(2,4,6-trifluorophenyl)[1,2,4]triazolo[1,5-a]-pyrimidine-7-amine
(22-4) 5-chloro-6-(2,4,6-trifluorophenyl)-7-(4-methylpiperidin-1-yl)[1,2,4]triazolo[1,5-a]pyrimidine
(23-1) 2-butoxy-6-iodo-3-propylbenzopyran-4-one
(23-2) 2-ethoxy-6-iodo-3-propylbenzopyran-4-one
(23-3) 6-iodo-2-propoxy-3-propylbenzopyran-4-one
(24-1) N-(3′,4′-dichloro-5-fluoro-1,1′-biphenyl-2-yl)-3-(difluoromethyl)-1-methyl-1H-pyrazole-4-carboxamide
(24-3) 3-(trifluoromethyl)-N-{3′-fluoro-4′-[(E)-(methoxyimino)methyl]-1,1′-biphenyl-2-yl}-1-methyl-1H-pyrazole-4-carboxamide
(24-7) N-(4′-bromo-1,1′-biphenyl-2-yl)-4-(difluoromethyl)-2-methyl-1,3-thiazole-5-carboxamide.

Very particularly preferred mixing partners are the following active compounds:
(2-2) fluoxastrobin
(2-4) trifloxystrobin
(2-3) (2E)-2-(2-{[6-(3-chloro-2-methylphenoxy)-5-fluoro-4-pyrimidinyl]oxy}phenyl)-2-(methoxyimino)-N-methylethanamide
(3-15) prothioconazole
(3-17) tebuconazole
(3-21) bitertanol
(3-22) triadimenol
(3-24) fluquinconazole
(4-1) dichlofluanid
(4-2) tolylfluanid
(5-1) iprovalicarb
(6-6) fenhexamid
(6-9) picobenzamid
(6-7) carpropamid
(6-14) penthiopyrad
(6-17) N-{2-[3-chloro-5-(trifluoromethyl)pyridin-2-yl]ethyl}-2-(trifluoromethyl)benzamide
(6b-2) N-(2-[1,1′-bicyclopropyl]-2-ylphenyl)-3-(difluoromethyl)-1-methyl-1H-pyrazole-4-carboxamide
(7-4) propineb
(8-4) metalaxyl-M
(8-5) benalaxyl-M
(9-3) pyrimethanil
(10-3) carbendazim
(11-4) propamocarb-fosetyl
(12-4) iprodione
(14-2) prochloraz
(14-3) triazoxide
(16-2) fludioxonil
(19-10) spiroxamine
(19-22) 2-(4-chlorophenyl)-N-{2-[3-methoxy-4-(prop-2-yn-1-yloxy)phenyl]ethyl}-2-(prop-2-yn-1-yloxy)acetamide
(22-4) 5-chloro-6-(2,4,6-trifluorophenyl)-7-(4-methylpiperidin-1-yl)[1,2,4]triazolo[1,5-a]pyrimidine
(24-1) N-(3′,4′-dichloro-5-fluoro-1,1′-biphenyl-2-yl)-3-(difluoromethyl)-1-methyl-1H-pyrazole-4-carboxamide.

Preferred active compound combinations comprising two groups of active compounds and in each case at least formononetin of the formula (I) and at least one active compound of the given group (2) to (24) are described below. These combinations are the active compound combinations A to U.

In addition to formononetin of the formula (I), the active compound combinations A also comprise a strobilurin of the formula (II) (group 2)

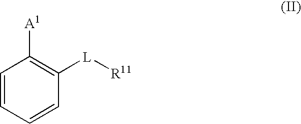

in which A¹, L and R¹¹ are as defined above.

Preferred are active compound combinations A in which the strobilurin of the formula (II) (group 2) is selected from the list below:
(2-1) azoxystrobin
(2-2) fluoxastrobin
(2-3) (2E)-2-(2-{[6-(3-chloro-2-methylphenoxy)-5-fluoro-4-pyrimidinyl]oxy}phenyl)-2-(methoxyimino)-N-methylethanamide
(2-4) trifloxystrobin
(2-5) (2E)-2-(methoxyimino)-N-methyl-2-(2-{[({(1E)-1-[3-(trifluoromethyl)phenyl]-ethyliden}amino)oxy]methyl}phenyl)ethanamide
(2-6) (2E)-2-(methoxyimino)-N-methyl-2-{2-[(E)-({1-[3-(trifluoromethyl)phenyl]ethoxy}-imino)methyl]phenyl}ethanamide
(2-7) orysastrobin
(2-8) 5-methoxy-2-methyl-4-(2-{[({(1E)-1-[3-(trifluoromethyl)phenyl]ethyliden}-amino)oxy]methyl}phenyl)-2,4-dihydro-3H-1,2,4-triazol-3-one
(2-9) kresoxim-methyl
(2-10) dimoxystrobin
(2-11) picoxystrobin
(2-12) pyraclostrobin
(2-13) metominostrobin

Particularly preferred are active compound combinations A in which the strobilurin of the formula (II) (group 2) is selected from the list below:
(2-1) azoxystrobin
(2-2) fluoxastrobin
(2-3) (2E)-2-(2-{[6-(3-chloro-2-methylphenoxy)-5-fluoro-4-pyrimidinyl]oxy}phenyl)-2-(methoxyimino)-N-methylethanamide
(2-4) trifloxystrobin
(2-12) pyraclostrobin
(2-9) kresoxim-methyl
(2-10) dimoxystrobin
(2-11) picoxystrobin
(2-13) metominostrobin

Emphasis is given to the active compound combinations A listed in Table 1 below:

TABLE 1
Active compound combinations A

| No. | | Strobilurin of the formula (II) |
|---|---|---|
| A-1 | formononetin | (2-2) fluoxastrobin |
| A-2 | formononetin | (2-3)(2E)-2-(2-{[6-(3-chloro-2- |
| | | methylphenoxy)-5-fluoro-4- |
| | | pyrimidinyl]oxy}phenyl)-2- |
| | | (methoxyimino)-N-methylethanamide |
| A-3 | formononetin | (2-4) trifloxystrobin |
| A-4 | formononetin | (2-1) azoxystrobin |
| A-5 | formononetin | (2-12) pyraclostrobin |
| A-6 | formononetin | (2-9) kresoxim-methyl |
| A-7 | formononetin | (2-10) dimoxystrobin |
| A-8 | formononetin | (2-11) picoxystrobin |
| A-9 | formononetin | (2-13) metominostrobin |

In addition to formononetin of the formula (I), the active compound combinations B also comprise a triazole of the formula (III) (group 3)

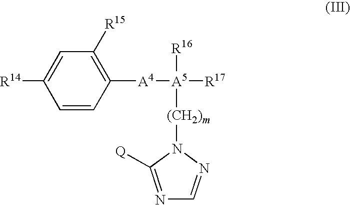

in which Q, m, R¹⁴, R¹⁵, A⁴, A⁵, R¹⁶ and R¹⁷ are as defined above.

Preference is given to active compound combinations B in which the triazole of the formula (III) (group 3) is selected from the list below:
(3-1) azaconazole
(3-2) etaconazole
(3-3) propiconazole
(3-4) difenoconazole
(3-5) bromuconazole
(3-6) cyproconazole
(3-7) hexaconazole
(3-8) penconazole
(3-9) myclobutanil
(3-10) tetraconazole
(3-11) flutriafol
(3-12) epoxiconazole
(3-13) flusilazole
(3-14) simeconazole
(3-15) prothioconazole
(3-16) fenbuconazole
(3-17) tebuconazole
(3-18) ipconazole
(3-19) metconazole
(3-20) triticonazole
(3-21) bitertanol
(3-22) triadimenol
(3-23) triadimefon
(3-24) fluquinconazole
(3-25) quinconazole

Particular preference is given to active compound combinations B in which the triazole of the formula (III) (group 3) is selected from the list below:
(3-3) propiconazole
(3-6) cyproconazole
(3-15) prothioconazole
(3-17) tebuconazole
(3-21) bitertanol
(3-4) difenoconazole
(3-7) hexaconazole
(3-19) metconazole
(3-22) triadimenol
(3-24) fluquinconazole

Emphasis is given to the active compound combinations B listed in Table 2 below:

TABLE 2
Active compound combinations B

| No. | | Triazole of the formula (III) |
|---|---|---|
| B-1 | formononetin | (3-3) propiconazole |
| B-2 | formononetin | (3-6) cyproconazole |
| B-3 | formononetin | (3-15) prothioconazole |
| B-4 | formononetin | (3-17) tebuconazole |
| B-1 | formononetin | (3-21) bitertanol |
| B-2 | formononetin | (3-4) difenoconazole |
| B-3 | formononetin | (3-7) hexaconazole |
| B-4 | formononetin | (3-19) metconazole |
| B-5 | formononetin | (3-22) triadimenol |
| B-6 | formononetin | (3-24) fluquinconazole |
| B-7 | formononetin | (3-4) difenoconazole |
| B-8 | formononetin | (3-7) hexaconazole |
| B-9 | formononetin | (3-19) metconazole |
| B-10 | formononetin | (3-22) triadimenol |

In addition to formononetin of the formula (I), the active compound combinations C also comprise a sulphenamide of the formula (IV) (group 4)

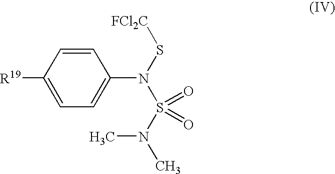

in which R¹⁹ is as defined above.

Preference is given to active compound combinations C in which the sulphenamide of the formula (N) (group 4) is selected from the list below:
(4-1) dichlofluanid
(4-2) tolylfluanid

Emphasis is given to the active compound combinations C listed in Table 3 below:

TABLE 3
Active compound combinations C

| No. | | Sulphenamide of the formula (IV) |
|---|---|---|
| C-1 | formononetin | (4-1) dichlofluanid |
| C-2 | formononetin | (4-2) tolylfluanid |

In addition to formononetin of the formula (I), the active compound combinations D also comprise a valinamide (group 5) selected from
(5-1) iprovalicarb
(5-2) N¹-[2-(4-{[3-(4-chlorophenyl)-2-propynyl]oxy}-3-methoxyphenyl)ethyl]-N²-(methyl-sulphonyl)-D-valinamide
(5-3) benthiavalicarb

Preference is given to active compound combinations D in which the valinamide (group 5) is selected from the list below:
(5-1) iprovalicarb
(5-3) benthiavalicarb

Emphasis is given to the active compound combinations D listed in Table 4 below:

TABLE 4
Active compound combinations D

| No. | | Valinamide |
|---|---|---|
| D-1 | formononetin | (5-1) iprovalicarb |
| D-2 | formononetin | (5-3) benthiavalicarb |

In addition to formononetin of the formula (I), the active compound combinations E also comprise a carboxamide of the formula (V) (group 6)

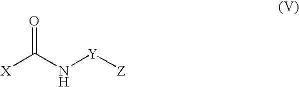

in which X, Y and Z are as defined above.

Preference is given to active compound combinations E in which the carboxamide of the formula (V) (group 6) is selected from the list below:
(6-1) 2-chloro-N-(1,1,3-trimethylindan-4-yl)nicotinamide
(6-2) boscalid
(6-3) furametpyr
(6-4) N-(3-p-tolylthiophene-2-yl)-1-methyl-3-trifluoromethyl-1H-pyrazole-4-carboxamide
(6-5) ethaboxam
(6-6) fenhexamid
(6-7) carpropamid
(6-8) 2-chloro-4-(2-fluoro-2-methylpropionylamino)-N,N-dimethylbenzamide
(6-9) picobenzamid
(6-10) zoxamide
(6-11) 3,4-dichloro-N-(2-cyanophenyl)isothiazole-5-carboxamide
(6-12) carboxin
(6-13) tiadinil
(6-14) penthiopyrad
(6-15) silthiofam
(6-16) N-[2-(1,3-dimethylbutyl)phenyl]-1-methyl-4-(trifluoromethyl)-1H-pyrrole-3-carboxamide
(6-17) N-{2-[3-chloro-5-(trifluoromethyl)pyridin-2-yl]ethyl}-2-(trifluoromethyl)benzamide
(6b-2) N-(2-[1,1′-bicyclopropyl]-2-ylphenyl)-3-(difluoromethyl)-1-methyl-1H-pyrazole-4-carboxamide

Particular preference is given to active compound combinations E in which the carboxamide of the formula (V) (group 6) is selected from the list below:
(6-2) boscalid
(6-5) ethaboxam
(6-6) fenhexamid
(6-7) carpropamid
(6-8) 2-chloro-4-(2-fluoro-2-methyl-propionylamino)-N,N-dimethylbenzamide
(6-9) picobenzamid
(6-10) zoxamide
(6-11) 3,4-dichloro-N-(2-cyanophenyl)isothiazole-5-carboxamide
(6-14) penthiopyrad
(6-16) N-[2-(1,3-dimethylbutyl)phenyl]-1-methyl-4-(trifluoromethyl)-1H-pyrrole-3-carboxamide

Very particular preference is given to active compound combinations E in which the carboxamide of the formula (V) (group 6) is selected from the list below:
(6-2) boscalid
(6-6) fenhexamid
(6-7) carpropamid
(6-9) picobenzamid
(6-14) penthiopyrad
(6-17) N-{2-[3-chloro-5-(trifluoromethyl)pyridin-2-yl]ethyl}-2-(trifluoromethyl)benzamide
(6b-2) N-(2-[1,1′-bicyclopropyl]-2-ylphenyl)-3-(difluoromethyl)-1-methyl-1H-pyrazole-4-carboxamide

Emphasis is given to the active compound combinations E listed in Table 5 below:

TABLE 5
Active compound combinations E

| No. | | Carboxamide of the formula (V) |
|---|---|---|
| E-1 | formononetin | (6-2) boscalid |
| E-2 | formononetin | (6-6) fenhexamid |
| E-3 | formononetin | (6-7) carpropamid |
| E-4 | formononetin | (6-9) picobenzamid |
| E-5 | formononetin | (6-14) penthiopyrad |

In addition to formononetin of the formula (I), the active compound combinations F also comprise a dithiocarbamate (group 7) selected from
(7-1) mancozeb
(7-2) maneb
(7-3) metiram
(7-4) propineb
(7-5) thiram
(7-6) zineb
(7-7) ziram

Preference is given to active compound combinations F in which the dithiocarbamate (group 7) is selected from the list below:
(7-1) mancozeb
(7-2) maneb
(7-4) propineb
(7-5) thiram
(7-6) zineb

Particular preference is given to active compound combinations F in which the dithiocarbamate (group 7) is selected from the list below:
(7-1) mancozeb
(7-4) propineb

Emphasis is given to the active compound combinations F listed in Table 6 below:

TABLE 6
Active compound combinations F

| No. | | Dithiocarbamate |
|---|---|---|
| F-1 | formononetin | (7-1) mancozeb |
| F-2 | formononetin | (7-4) propineb |

In addition to formononetin of the formula (I), the active compound combinations G also comprise an acylalanine of the formula (VI) (group 8)

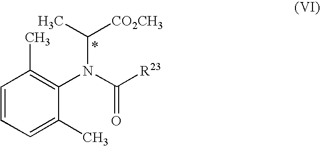

in which * and R²³ are as defined above.

Preference is given to active compound combinations G in which the acylalanine of the formula (VI) (group 8) is selected from the list below:
(8-1) benalaxyl
(8-2) furalaxyl
(8-3) metalaxyl
(8-4) metalaxyl-M
(8-5) benalaxyl-M

Particular preference is given to active compound combinations G in which the acylalanine of the formula (VI) (group 8) is selected from the list below:
(8-3) metalaxyl
(8-4) metalaxyl-M
(8-5) benalaxyl-M

Emphasis is given to the active compound combinations G listed in Table 7 below:

TABLE 7
Active compound combinations G

| No. | | Acylalanine of the formula (VI) |
|---|---|---|
| G-1 | formononetin | (8-3) metalaxyl |
| G-2 | formononetin | (8-4) metalaxyl-M |
| G-3 | formononetin | (8-5) benalaxyl-M |

In addition to formononetin of the formula (I), the active compound combinations H also comprise an anilinopyrimidine (group 9) selected from
(9-1) cyprodinil
(9-2) mepanipyrim
(9-3) pyrimethanil

Emphasis is given to the active compound combinations H listed in Table 8 below:

TABLE 8
Active compound combinations H

| No. | | Anilinopyrimidine |
|---|---|---|
| H-1 | formononetin | (9-1) cyprodinil |
| H-2 | formononetin | (9-2) mepanipyrim |
| H-3 | formononetin | (9-3) pyrimethanil |

In addition to formononetin of the formula (I), the active compound combinations I also comprise a benzimidazole of the formula (VIII) (group 10)

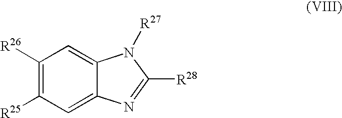

in which R²⁵, R²⁶, R²⁷ and R²⁸ are as defined above.

Preference is given to active compound combinations I in which the benzimidazole of the formula (VIII) (group 10) is selected from the list below:
(10-1) 6-chloro-5-[(3,5-dimethylisoxazol-4-yl)sulphonyl]-2,2-difluoro-5H-[1,3]dioxolo[4,5-f]-benzimidazole
(10-2) benomyl
(10-3) carbendazim
(10-4) chlorfenazole
(10-5) fuberidazole
(10-6) thiabendazole

Particular preference is given to active compound combinations I in which the benzimidazole of the formula (VIII) (group 10) is:
(10-3) carbendazim

Emphasis is given to the active compound combinations I listed in Table 9 below:

TABLE 9
Active compound combinations I

| No. | | Benzimidazole of the formula (VIII) |
|---|---|---|
| I-1 | formononetin | (10-3) carbendazim |

In addition to formononetin of the formula (I), the active compound combinations J also comprise a carbamate (group 11) of the formula (IX)

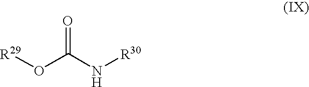

in which R²⁹ and R³⁰ are as defined above.

Preference is given to active compound combinations J in which the carbamate (group 11) is selected from the list below:
(11-1) diethofencarb
(11-2) propamocarb
(11-3) propamocarb-hydrochloride
(11-4) propamocarb-fosetyl

Emphasis is given to the active compound combinations J listed in Table 10 below:

TABLE 10
Active compound combinations J

| No. | | Carbamate of the formula (IX) |
|---|---|---|
| J-1 | formononetin | (11-2) propamocarb |
| J-2 | formononetin | (11-3) propamocarb-hydrochloride |
| J-3 | formononetin | (11-4) propamocarb-fosetyl |

In addition to formononetin of the formula (I), the active compound combinations K also comprise a dicarboximide (group 12) selected from
(12-1) captafol
(12-2) captan
(12-3) folpet
(12-4) iprodione
(12-5) procymidone
(12-6) vinclozolin

Preference is given to active compound combinations K in which the dicarboximide (group 12) is selected from the list below:
(12-2) captan
(12-3) folpet
(12-4) iprodione

Emphasis is given to the active compound combinations K listed in Table 11 below:

TABLE 11
Active compound combinations K

| No. | | Dicarboximide |
|---|---|---|
| K-1 | formononetin | (12-2) captan |
| K-2 | formononetin | (12-3) folpet |
| K-3 | formononetin | (12-4) iprodione |

In addition to formononetin of the formula (I), the active compound combinations L also comprise a guanidine (group 13) selected from
(13-1) dodine
(13-2) guazatine
(13-3) iminoctadine triacetate
(13-4) iminoctadine tris(albesilate)

Preference is given to active compound combinations L in which the guanidine (group 13) is selected from the list below:
(13-1) dodine
(13-2) guazatine

Emphasis is given to the active compound combinations L listed in Table 12 below:

TABLE 12
Active compound combinations L

| No. | | Guanidine |
|---|---|---|
| L-1 | formononetin | (13-1) dodine |
| L-2 | formononetin | (13-2) guazatine |

In addition to formononetin of the formula (I), the active compound combinations M also comprise an imidazole (group 14) selected from
(14-1) cyazofamid
(14-2) prochloraz
(14-3) triazoxide
(14-4) pefurazoate

Preference is given to active compound combinations M in which the imidazole (group 14) is selected from the list below:
(14-2) prochloraz
(14-3) triazoxide

Emphasis is given to the active compound combinations M listed in Table 13 below:

TABLE 13
Active compound combinations M

| No. | | Imidazole |
|---|---|---|
| M-1 | formononetin | (14-2) prochloraz |
| M-2 | formononetin | (14-3) triazoxide |

In addition to formononetin of the formula (I), the active compound combinations N also comprise a morpholine (group 15) of the formula (X)

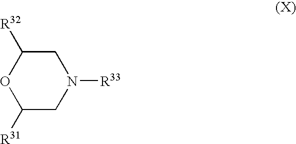

in which R³¹, R³² and R³³ are as defined above.

Preference is given to active compound combinations N in which the morpholine (group 15) of the formula (X) is selected from the list below:
(15-1) aldimorph
(15-2) tridemorph
(15-3) dodemorph
(15-4) fenpropimorph
(15-5) dimethomorph

Particular preference is given to active compound combinations N in which the morpholine (group 15) of the formula (X) is selected from the list below:
(15-4) fenpropimorph
(15-5) dimethomorph

Emphasis is given to the active compound combinations N listed in Table 14 below:

TABLE 14
Active compound combinations N

| No. | | Morpholine of the formula (X) |
|---|---|---|
| N-1 | formononetin | (15-4) fenpropimorph |

In addition to formononetin of the formula (I), the active compound combinations O also comprise a pyrrole (group 16) of the formula (XI)

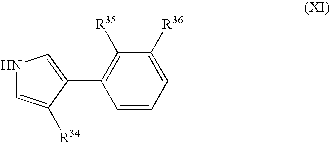

in which R³⁴, R³⁵ and R³⁶ are as defined above.

Preference is given to active compound combinations O in which the pyrrole (group 16) of the formula (XI) is selected from the list below:
(16-1) fenpiclonil
(16-2) fludioxonil
(16-3) pyrrolnitrin

Particular preference is given to active compound combinations O in which the pyrrole (group 16) of the formula (XI) is selected from the list below:
(16-2) fludioxonil

Emphasis is given to the active compound combinations O listed in Table 15 below:

TABLE 15
Active compound combinations O

| No. | | Pyrrole of the formula (XI) |
|---|---|---|
| O-1 | formononetin | (16-2) fludioxonil |

In addition to formononetin of the formula (I), the active compound combinations P also comprise a phosphonate (group 17) selected from
(17-1) fosetyl-A1
(17-2) phosphonic acid

Emphasis is given to the active compound combinations P listed in Table 16 below:

TABLE 16
Active compound combinations P

| No. | | Phosphonate |
|---|---|---|
| P-1 | formononetin | (17-1) fosetyl-Al |

In addition to formononetin of the formula (I), the active compound combinations Q also comprise a fungicide (group 19) selected from
(19-1) acibenzolar-S-methyl
(19-2) chlorothalonil
(19-3) cymoxanil
(19-4) edifenphos
(19-5) famoxadone
(19-6) fluazinam
(19-7) copper oxychloride
(19-8) copper hydroxide
(19-9) oxadixyl
(19-10) spiroxamine
(19-11) dithianon
(19-12) metrafenone
(19-13) fenamidone
(19-14) 2,3-dibutyl-6-chlorothieno[2,3-d]pyrimidin-4(3H)-one
(19-15) probenazole
(19-16) isoprothiolane
(19-17) kasugamycin
(19-18) phthalide
(19-19) ferimzone
(19-20) tricyclazole
(19-21) N-({4-[(cyclopropylamino)carbonyl]phenyl}sulphonyl)-2-methoxybenzamide
(19-22) 2-(4-chlorophenyl)-N-{2-[3-methoxy-4-(prop-2-yn-1-yloxy)phenyl]ethyl}-2-(prop-2-yn-1-yloxy)acetamide

Preference is given to active compound combinations Q in which the fungicide (group 19) is selected from the list below:
(19-1) acibenzolar-S-methyl
(19-2) chlorothalonil
(19-3) cymoxanil
(19-5) famoxadone
(19-6) fluazinam
(19-7) copper oxychloride
(19-9) oxadixyl
(19-10) spiroxamine
(19-13) fenamidone
(19-21) N-({4-[(cyclopropylamino)carbonyl]phenyl}sulphonyl)-2-methoxybenzamide
(19-22) 2-(4-chlorophenyl)-N-{2-[3-methoxy-4-(prop-2-yn-1-yloxy)phenyl]ethyl}-2-(prop-2-yn-1-yloxy)acetamide

Particular preference is given to active compound combinations Q in which the fungicide (group 19) is selected from the following list:
(19-2) chlorothalonil
(19-7) copper oxychloride
(19-10) spiroxamine
(19-21) N-({4-[(cyclopropylamino)carbonyl]phenyl}sulphonyl)-2-methoxybenzamide
(19-22) 2-(4-chlorophenyl)-N-{2-[3-methoxy-4-(prop-2-yn-1-yloxy)phenyl]ethyl}-2-(prop-2-yn-1-yloxy)acetamide

Emphasis is given to the active compound combinations Q listed in Table 17 below:

TABLE 17
Active compound combinations Q

| No. | | Fungicide |
|---|---|---|
| Q-1 | formononetin | (19-2) chlorothalonil |
| Q-2 | formononetin | (19-7) copper oxychloride |
| Q-3 | formononetin | (19-10) spiroxamine |
| Q-4 | formononetin | (19-21) N-({4-[(cyclopropylamino)carbonyl]- |
| | | phenyl}sulphonyl)-2- |
| | | methoxybenzamide |
| Q-5 | formononetin | (19-22) 2-(4-chlorophenyl)-N-{2-[3- |
| | | methoxy-4-(prop-2-yn-1-yloxy)- |
| | | phenyl]ethyl}-2-(prop-2-yn-1- |
| | | yloxy)acetamide |

In addition to formononetin of the formula (I), the active compound combinations R also comprise a (thio)urea derivative (group 20) selected from
(20-1) pencycuron
(20-2) thiophanate-methyl
(20-3) thiophanate-ethyl

Preference is given to active compound combinations R in which the (thio)urea derivative (group 20) is selected from the list below:
(20-1) pencycuron
(20-2) thiophanate-methyl

Emphasis is given to the active compound combinations R listed in Table 18 below:

TABLE 18
Active compound combinations R

| No. | | (Thio)urea derivative |
|---|---|---|
| R-1 | formononetin | (20-1) pencycuron |

In addition to formononetin of the formula (I) the active compound combinations S also comprise a triazolopyrimidine (group 22) of the formula (XIV)

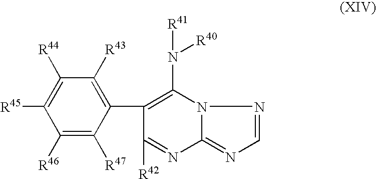

in which R⁴⁰, R⁴¹, R⁴², R⁴³, R⁴⁴, R⁴⁵, R⁴⁶ and R⁴⁷ are as defined above.

Preference is given to active compound combinations S in which the triazolopyrimidine (group 22) of the formula (XIV) is selected from the list below:
(22-1) 5-chloro-N-[(1S)-2,2,2-trifluoro-1-methylethyl]-6-(2,4,6-trifluorophenyl)[1,2,4]triazolo-[1,5-a]pyrimidine-7-amine
(22-2) 5-chloro-N-[(1R)-1,2-dimethylpropyl]-6-(2,4,6-trifluorophenyl)[1,2,4]triazolo[1,5-a]-pyrimidine-7-amine
(22-3) 5-chloro-6-(2-chloro-6-fluorophenyl)-7-(4-methylpiperidin-1-yl)[1,2,4]triazolo[1,5-a]-pyrimidine
(22-4) 5-chloro-6-(2,4,6-trifluorophenyl)-7-(4-methylpiperidin-1-yl)[1,2,4]triazolo[1,5-a]pyrimidine

Particular preference is given to active compound combinations S in which the triazolopyrimidine (group 22) of the formula (XIV) is selected from the list below:
(22-1) 5-chloro-N-[(1S)-2,2,2-trifluoro-1-methylethyl]-6-(2,4,6-trifluorophenyl)[1,2,4]triazolo-[1,5-a]pyrimidine-7-amine
(22-2) 5-chloro-N-[(1R)-1,2-dimethylpropyl]-6-(2,4,6-trifluorophenyl)[1,2,4]triazolo[1,5-a]-pyrimidine-7-amine
(22-4) 5-chloro-6-(2,4,6-trifluorophenyl)-7-(4-methylpiperidin-1-yl)[1,2,4]triazolo[1,5-a]pyrimidine

Emphasis is given to the active compound combinations S listed in Table 19 below:

TABLE 19
Active compound combinations S

| No. | | Triazolopyrimidine of the formula (XIV) |
|---|---|---|
| S-1 | formononetin | (22-1) 5-chloro-N-[(1S)-2,2,2-trifluoro- |
| | | 1-methylethyl]-6-(2,4,6- |
| | | trifluorophenyl)[1,2,4]triazolo- |
| | | [1,5-a]pyrimidine-7-amine |
| S-2 | formononetin | (22-2) 5-chloro-N-[(1R)-1,2- |
| | | dimethylpropyl]-6-(2,4,6- |
| | | trifluorophenyl)[1,2,4]triazolo- |
| | | [1,5-a]pyrimidine-7-amine |
| S-3 | formononetin | (22-4) 5-chloro-6-(2,4,6-trifluorophenyl)- |
| | | 7-(4-methylpiperidin-1- |
| | | yl)[1,2,4]triazolo[1,5-a]pyrimidine |

In addition to formononetin of the formula (I), the active compound combinations T also comprise an iodochromone (group 23) of the formula (XV)

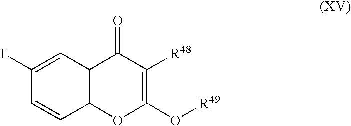

in which R⁴⁸ and R⁴⁹ are as defined above.

Preference is given to active compound combinations T in which the iodochromone (group 23) of the formula (XV) is selected from the list below:
(23-1) 2-butoxy-6-iodo-3-propylbenzopyran-4-one
(23-2) 2-ethoxy-6-iodo-3-propylbenzopyran-4-one
(23-3) 6-iodo-2-propoxy-3-propylbenzopyran-4-one
(23-4) 2-but-2-ynyloxy-6-iodo-3-propylbenzopyran-4-one
(23-5) 6-iodo-2-(1-methylbutoxy)-3-propylbenzopyran-4-one
(23-6) 2-but-3-enyloxy-6-iodobenzopyran-4-one
(23-7) 3-butyl-6-iodo-2-isopropoxybenzopyran-4-one

Particular preference is given to active compound combinations T in which the iodochromone (group 23) of the formula (XV) is selected from the list below:
(23-1) 2-butoxy-6-iodo-3-propylbenzopyran-4-one
(23-2) 2-ethoxy-6-iodo-3-propylbenzopyran-4-one

Emphasis is given to the active compound combinations T listed in Table 20 below:

TABLE 20
Active compound combinations T

| No. | | Iodochromone of the formula (XV) |
|---|---|---|
| T-1 | formononetin | (23-1) 2-butoxy-6-iodo-3-propyl-benzopyran- |
| | | 4-one |
| T-2 | formononetin | (23-2) 2-ethoxy-6-iodo-3-propyl-benzopyran- |
| | | 4-one |

In addition to formononetin of the formula (I), the active compound combinations U also comprise a biphenylcarboxamide (group 24) of the formula (XVI)

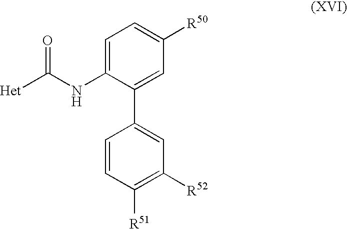

in which R⁵⁰, R⁵¹, R⁵² and Het are as defined above.

Preference is given to active compound combinations U in which the biphenylcarboxamide (group 24) of the formula (XVI) is selected from the list below:
(24-1) N-(3′,4′-dichloro-5-fluoro-1,1′-biphenyl-2-yl)-3-(difluoromethyl)-1-methyl-1H-pyrazole-4-carboxamide
(24-2) 3-(difluoromethyl)-N-{3′-fluoro-4′-[(E)-(methoxyimino)methyl]-1,1′-biphenyl-2-yl}-1-methyl-1H-pyrazole-4-carboxamide
(24-3) 3-(trifluoromethyl)-N-{3′-fluoro-4′-[(E)-(methoxyimino)methyl]-1,1′-biphenyl-2-yl}-1-methyl-1H-pyrazole-4-carboxamide
(24-4) N-(3′,4′-dichloro-1,1′-biphenyl-2-yl)-5-fluoro-1,3-dimethyl-1H-pyrazole-4-carboxamide
(24-5) N-(4′-chloro-3′-fluoro-1,1′-biphenyl-2-yl)-2-methyl-4-(trifluoromethyl)-1,3-thiazole-5-carboxamide
(24-6) N-(4′-chloro-1,1′-biphenyl-2-yl)-4-(difluoromethyl)-2-methyl-1,3-thiazole-5-carboxamide
(24-7) N-(4′-bromo-1,1′-biphenyl-2-yl)-4-(difluoromethyl)-2-methyl-1,3-thiazole-5-carboxamide
(24-8) 4-(difluoromethyl)-2-methyl-N-[4′-(trifluoromethyl)-1,1′-biphenyl-2-yl]-1,3-thiazole-5-carboxamide.

Particular preference is given to active compound combinations U in which the biphenylcarboxamide (group 24) of the formula (XVI) is selected from the list below:
(24-1) N-(3′,4′-dichloro-5-fluoro-1,1′-biphenyl-2-yl)-3-(difluoromethyl)-1-methyl-1H-pyrazole-4-carboxamide
(24-3) 3-(trifluoromethyl)-N-{3′-fluoro-4′-[(E)-(methoxyimino)methyl]-1,1′-biphenyl-2-yl}-1-methyl-1H-pyrazole-4-carboxamide
(24-7) N-(4′-bromo-1,1′-biphenyl-2-yl)-4-(difluoromethyl)-2-methyl-1,3-thiazole-5-carboxamide

Emphasis is given to the active compound combinations U listed in Table 21 below:

TABLE 21
Active compound combinations U

| No. | | Biphenylcarboxamide of the formula (XVI) |
|---|---|---|
| U-1 | formononetin | (24-1) N-(3′,4′-dichloro-5-fluoro-1,1′- |
| | | biphenyl-2-yl)-3-(difluoromethyl)-1- |
| | | methyl-1H-pyrazole-4-carboxamide |
| U-2 | formononetin | (24-3) 3-(trifluoromethyl)-N-{3′-fluoro- |
| | | 4′-[(E)-(methoxyimino)methyl]-1,1′- |
| | | biphenyl-2-yl}-1-methyl-1H-pyrazole- |
| | | 4-carboxamide |
| U-3 | formononetin | (24-7) N-(4′-bromo-1,1′-biphenyl-2-yl)- |
| | | 4-(difluoromethyl)-2-methyl-1,3- |
| | | thiazole-5-carboxamide |

In addition to formononetin of the formula (I), the active compound combinations according to the invention comprise at least one active compound from the compounds of groups (2) to (24). In addition, they may also comprise further fungicidally active additives.

If the active compounds in the active compound combinations according to the invention are present in certain weight ratios, the synergistic effect is particularly pronounced. However, the weight ratios of the active compounds in the active compound combinations can be varied within a relatively wide range. In general, the active compound combinations according to the invention comprise active formononetin and a mixing partner from one of the groups (2) to (24) in the mixing ratios listed in an exemplary manner in Table 22 below.

The mixing ratios are based on ratios by weight. The ratio is to be understood as active compound formononetin of the formula (I): mixing partner.

Preferred are mixing ratios between 50,000:1 to 1:50,000

Particularly preferred are ratios between 10,000:1 to 1:10,000

Even more preferred mixing ratios are listed in Table 22.

TABLE 22
Mixing ratios

| | Very preferred | Most preferred |
|---|---|---|
| Mixing partner | mixing ratio | mixing ratio |
| Group (2): strobilurins | 50:1 to 1:50 | 10:1 to 1:20 |
| Group (3): triazoles except for (3-15) | 50:1 to 1:50 | 20:1 to 1:20 |
| (3-15): prothioconazole | 50:1 to 1:50 | 10:1 to 1:20 |
| Group (4): sulphenamides | 1:1 to 1:150 | 1:1 to 1:100 |
| Group (5): valinamides | 50:1 to 1:50 | 10:1 to 1:20 |
| Group (6): carboxamides | 50:1 to 1:50 | 20:1 to 1:20 |
| Group (7): dithiocarbamates | 1:1 to 1:150 | 1:1 to 1:100 |
| Group (8): acylalanines | 10:1 to 1:150 | 5:1 to 1:100 |
| Group (9): anilinopyrimidines | 5:1 to 1:50 | 1:1 to 1:20 |
| Group (10): benzimidazoles | 10:1 to 1:50 | 5:1 to 1:20 |
| Group (11): carbamates except | 1:1 to 1:150 | 1:1 to 1:100 |
| for (11-1) | | |
| (11-1): diethofencarb | 50:1 to 1:50 | 10:1 to 1:20 |
| Group (12): (12-1)/(12-2)/(12-3) | 1:1 to 1:150 | 1:5 to 1:100 |
| Group (12): (12-4)/(12-5)/(12-6) | 5:1 to 1:50 | 1:1 to 1:20 |
| Group (13): guanidines | 100:1 to 1:150 | 20:1 to 1:100 |
| Group (14): imidazoles | 50:1 to 1:50 | 10:1 to 1:20 |
| Group (15): morpholines | 50:1 to 1:50 | 10:1 to 1:20 |
| Group (16): pyrroles | 50:1 to 1:50 | 10:1 to 1:20 |
| Group (17): phosphonates | 10:1 to 1:150 | 1:1 to 1:100 |
| Group (18): phenylethanamides | 50:1 to 1:50 | 10:1 to 1:20 |
| (19-1): acibenzolar-S-methyl | 50:1 to 1:50 | 20:1 to 1:20 |
| (19-2): chlorothalonil | 1:1 to 1:150 | 1:1 to 1:100 |
| (19-3): cymoxanil | 10:1 to 1:50 | 5:1 to 1:20 |
| (19-4): edifenphos | 10:1 to 1:50 | 5:1 to 1:20 |
| (19-5): famoxadone | 50:1 to 1:50 | 10:1 to 1:20 |
| (19-6): fluazinam | 50:1 to 1:50 | 10:1 to 1:20 |
| (19-7): copper oxychloride | 1:1 to 1:150 | 1:5 to 1:100 |
| (19-8): copper hydroxide | 1:1 to 1:150 | 1:5 to 1:100 |
| (19-9): oxadixyl | 10:1 to 1:150 | 5:1 to 1:100 |
| (19-10): spiroxamine | 50:1 to 1:50 | 10:1 to 1:20 |
| (19-11) dithianon | 50:1 to 1:50 | 10:1 to 1:20 |
| (19-12) metrafenone | 50:1 to 1:50 | 10:1 to 1:20 |
| (19-13) fenamidone | 50:1 to 1:50 | 10:1 to 1:20 |
| (19-14): 2,3-dibutyl-6-chlorothieno- | 50:1 to 1:50 | 10:1 to 1:20 |
| [2,3-d]pyrimidin-4(3H)one | | |
| (19-15): probenazole | 10:1 to 1:150 | 5:1 to 1:100 |
| (19-16): isoprothiolane | 10:1 to 1:150 | 5:1 to 1:100 |
| (19-17): kasugamycin | 50:1 to 1:50 | 10:1 to 1:20 |
| (19-18): phthalide | 10:1 to 1:150 | 5:1 to 1:100 |
| (19-19): ferimzone | 50:1 to 1:50 | 10:1 to 1:20 |
| (19-20): tricyclazole | 50:1 to 1:50 | 10:1 to 1:20 |
| (19-21): N-({4-[(cyclopropylamino)- | 10:1 to 1:150 | 5:1 to 1:100 |
| carbonyl]phenyl}sulphonyl)-2- | | |
| methoxybenzamide | | |
| (19-22) 2-(4-chlorophenyl)-N-{2- | 50:1 to 1:50 | 10:1 to 1:20 |
| [3-methoxy-4-(prop-2-yn-1- | | |
| yloxy)phenyl]ethyl}-2-(prop- | | |
| 2-yn-1-yloxy)acetamide | | |
| Group (20): (thio)urea derivatives | 50:1 to 1:50 | 10:1 to 1:20 |
| Group (21): amides | 50:1 to 1:50 | 10:1 to 1:20 |
| Group (22): triazolopyrimidines | 50:1 to 1:50 | 10:1 to 1:20 |
| Group (23): iodochromones | 50:1 to 1:50 | 10:1 to 1:20 |
| Group (24): biphenylcarboxamides | 50:1 to 1:50 | 10:1 to 1:20 |

In each case, the mixing ratio is to be chosen such that a synergistic mixture is obtained. The mixing ratios between the compound of the formula (I) and a compound of one of the groups (2) to (24) may also vary between the individual compounds of a group.

The active compound combinations according to the invention have very good fungicidal properties and are suitable for controlling phytopathogenic fungi, such as Plasmodiophoromycetes, Oomycetes, Chytridiomycetes, Zygomycetes, Ascomycetes, Basidiomycetes, Deuteromycetes, etc.

The active compound combinations according to the invention are particularly suitable for controlling Erysiphe graminis, Pyrenophora teres and Leptosphaeria nodorum.

Some pathogens causing fungal diseases which come under the generic names listed above may be mentioned by way of example, but not by way of limitation:

Pythium species, such as, for example, Pythium ultimum; Phytophthora species, such as, for example, Phytophthora infestans; Pseudoperonospora species, such as, for example, Pseudoperonospora humuli or Pseudoperonospora cubensis; Plasmopara species, such as, for example, Plasmopara viticola; Bremia species, such as, for example, Bremia lactucae; Peronospora species, such as, for example, Peronospora pisi or P. brassicae; Erysiphe species, such as, for example, Erysiphe graminis; Sphaerotheca species, such as, for example, Sphaerotheca fuliginea; Podosphaera species, such as, for example, Podosphaera leucotricha; Venturia species, such as, for example, Venturia inaequalis; Pyrenophora species, such as, for example, Pyrenophora teres or P. graminea (conidia form: Drechslera, syn: Helminthosporium); Cochliobolus species, such as, for example, Cochliobolus sativus (conidia form: Drechslera, syn: Helminthosporium); Uromyces species, such as, for example, Uromyces appendiculatus; Puccinia species, such as, for example, Puccinia recondita; Sclerotinia species, such as, for example, Sclerotinia sclerotiorum; Tilletia species, such as, for example, Tilletia caries; Ustilago species, such as, for example, Ustilago nuda or Ustilago avenae; Pellicularia species, such as, for example, Pellicularia sasakii; Pyricularia species, such as, for example, Pyricularia oryzae; Fusarium species, such as, for example, Fusarium culmorum; Botrytis species, such as, for example, Botrytis cinerea; Septoria species, such as, for example, Septoria nodorum; Leptosphaeria species, such as, for example, Leptosphaeria nodorum; Cercospora species, such as, for example, Cercospora canescens; Alternaria species, such as, for example, Alternaria brassicae; Pseudocercosporella species, such as, for example, Pseudocercosporella herpotrichoides, Rhizoctonia species, such as, for example, Rhizoctonia solani.

The fact that the active compound combinations are well tolerated by plants at the concentrations required for controlling plant diseases permits a treatment of entire plants (above-ground parts of plants and roots), of propagation stock and seed, and of the soil. The active compound combinations according to the invention can be used for foliar application or else as seed dressings.

The fact that the active compounds which can be used are well tolerated by plants at the concentrations required for controlling plant diseases permits a treatment of the seed. Accordingly, the active compounds according to the invention can be used as seed dressings.

A large part of the damage to crop plants which is caused by phytopathogenic fungi occurs as early as when the seed is attacked during storage and after the seed is introduced into the soil, during and immediately after germination of the plants. This phase is particularly critical since the roots and shoots of the growing plant are particularly sensitive and even minor damage can lead to the death of the whole plant. Protecting the seed and the germinating plant by the use of suitable compositions is therefore of particularly great interest.

The control of phytopathogenic fungi which damage plants post-emergence is carried out primarily by treating the soil and the above-ground parts of plants with crop protection agents. Owing to the concerns regarding a possible impact of crop protection agents on the environment and the health of man and animals, there are efforts to reduce the amount of active compounds applied.

The control of phytopathogenic fungi by treating the seeds of plants has been known for a long time and is subject-matter of continuous improvements. However, the treatment of seed frequently entails a series of problems which cannot always be solved in a satisfactory manner. Thus, it is desirable to develop methods for protecting the seed and the germinating plant which dispense with the additional application of crop protection agents after sowing or after the emergence of the plants or where additional applications are at least reduced. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore in particular also relates to a method for the protection of seed and germinating plants from attack by phytopathogenic fungi, by treating the seed with a composition according to the invention.

The invention likewise relates to the use of the compositions according to the invention for the treatment of seed for protecting the seed and the germinating plant from phytopathogenic fungi.

Furthermore, the invention relates to seed which has been treated with a composition according to the invention so as to afford protection from phytopathogenic fungi.

One of the advantages of the present invention is that the particular systemic properties of the compositions according to the invention mean that treatment of the seed with these compositions not only protects the seed itself, but also the resulting plants after emergence, from phytopathogenic fungi. In this manner, the immediate treatment of the crop at the time of sowing or shortly thereafter can be dispensed with.

Furthermore, it must be considered as advantageous that the mixtures according to the invention can also be employed in particular in transgenic seed.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawn and ornamental plants. The treatment of seed of cereals (such as wheat, barley, rye and oats), maize and rice is of particular importance.

In the context of the present invention, the composition according to the invention is applied to the seed either alone or in a suitable formulation. Preferably, the seed is treated in a state which is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, for example, it is possible to use seed which has been harvested, cleaned and dried to a moisture content of below 15% by weight. Alternatively, it is also possible to use seed which, after drying, has, for example, been treated with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the composition to the seed in the form of a suitable formulation. Suitable formulations and methods for the treatment of seed are known to the skilled worker and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations according to the invention are also suitable for increasing the yield of crops. In addition, they show reduced toxicity and are well tolerated by plants.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to custom-ary treatment methods, for example by dipping, spraying, evaporating, atomizing, broadcasting, brushing-on and, in the case of propagation material, in particular in the case of seeds, furthermore by one- or multilayer coating.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defense of the plants against insects, by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are furthermore particularly emphasized are the increased tolerance of the plants to certain herbicidally active compounds, for example imidazolinones, sulphonylureas, glyphosate or phosphinothricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars which have these genetic traits or genetic traits still to be developed, and which will be developed and/or marketed in the future.

Depending on their particular physical and/or chemical properties, the active compound combinations according to the invention can be converted into the customary formulations, such as solutions, emulsions, suspensions, powders, dusts, foams, pastes, soluble powders, granules, aerosols, suspoemulsion concentrates, natural and synthetic materials impregnated with active compound and microencapsulations in polymeric substances and in coating compositions for seeds, and ULV cool and warm fogging formulations.

These formulations are produced in a known manner, for example by mixing the active compounds or active compound combinations with extenders, that is liquid solvents, liquefied gases under pressure, and/or solid carriers, optionally with the use of surfactants, that is emulsifiers and/or dispersants, and/or foam formers.

If the extender used is water, it is also possible to employ, for example, organic solvents as auxiliary sol-vents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water.

Liquefied gaseous extenders or carriers are to be understood as meaning liquids which are gaseous at standard temperature and under atmospheric pressure, for example aerosol propellants such as butane, propane, nitrogen and carbon dioxide.

Suitable solid carriers are: for example ammonium salts and ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The active compound content of the use forms prepared from the commercial formulations may be varied within wide ranges. The concentration of active compound of the use forms for controlling animal pests, such as insects and acarids, may be from 0.0000001 to 95% by weight of active compound and is preferably from 0.0001 to 1% by weight. Application is in a manner adapted to the use forms.

The formulations for controlling unwanted phytopathogenic fungi generally comprise between 0.1 and 95 percent by weight of active compound, preferably between 0.5 and 90%.

The active compound combinations according to the invention can be used as such, in the form of their formulations or as the use forms prepared therefrom, such as ready-to-use solutions, emulsifiable concentrates, emulsions, suspensions, wettable powders, soluble powders, dusts and granules. They are used in a customary manner, for example by watering (drenching), drip irrigation, spraying, atomizing, broadcasting, dusting, foaming, spreading-on, and as a powder for dry seed treatment, a solution for seed treatment, a water-soluble powder for seed treatment, a water-soluble powder for slurry treatment, or by encrusting.

The active compound combinations according to the invention can, in commercial formulations and in the use forms prepared from these formulations, be present as a mixture with other active compounds, such as insecticides, attractants, sterilants, bactericides, acaricides, nematicides, fungicides, growth regulators or herbicides.

When using the active compound combinations according to the invention, the application rates can be varied within a relatively wide range, depending on the kind of application. In the treatment of parts of plants, the application rates of active compound combination are generally between 0.1 and 10 000 g/ha, preferably between 10 and 1000 g/ha. In the treatment of seeds, the application rates of active compound combination are generally between 0.001 and 50 g per kilogram of seed, preferably between 0.01 and 10 g per kilogram of seed. In the treatment of the soil, the application rates of active compound combination are generally between 0.1 and 10 000 g/ha, preferably between 1 and 5000 g/ha.

The active compound combinations can be used as such, in the form of concentrates or in the form of generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if desired desiccants and UV stabilizers, and, if desired, colorants and pigments and other processing auxiliaries.

The inventive method for the protection of seeds and plants, arising from these seeds, against fungal diseases comprises a procedure in which the seed is treated at the same time with formononetin of formula (I) and at least one fungicide selected from groups (2) to (24). It further comprises a method in which the seed is treated with formononetin of formula (I) and at least one fungicide selected from groups (2) to (24) separately.

The invention also comprises a seed, which has been treated with formononetin of formula (I) and at least one fungicide selected from groups (2) to (24) at the same time. The invention also comprises a seed, which has been treated with formononetin of formula (I) and at least one fungicide selected from groups (2) to (24) separately. For such a seed, the active ingredients, can be applied in separate layers. These layers can optionally be separated by one or more additional layers that may or may not contain active ingredients.

### Insecticides/acaricides/nematicides:

The inventive compound combinations comprising formononetin and at least one fungicide can also be combined with insecticides as:

### 1 Acetylcholinesterase (AChE) Inhibitors

1.1 carbamates (for example alanycarb, aldicarb, aldoxycarb, allyxycarb, aminocarb, aza-methiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-s-methyl, demeton-s-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2 Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopennethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))
2.2 oxadiazines (for example indoxacarb)

### 3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)
3.2 nicotine, bensultap, cartap

### 4. Acetylcholine Receptor Modulators

4.1 spinosyns (for example spinosad)

### 5. Antagonists of GABA-Gated Chloride Channels

5.1 cyclodiene organochlorines (for example camphechlor, chlordane, endosulfan, gamma-HCH, HCH, heptachlor, lindane, methoxychlor)
5.2 fiproles (for example acetoprole, ethiprole, fipronil, vaniliprole)

### 6. Chloride Channel Activators

6.1 mectins (for example abamectin, avermectin, emamectin, emamectin-benzoate, ivermectin, milbemectin, milbemycin)

### 7. Juvenile Hormone Mimetics

(for example diofenolan, epofenonane, fenoxycarb, hydroprene, kinoprene, methoprene, pyriproxifen, triprene)

### 8. Ecdyson Agonists/Disruptors

8.1 diacylhydrazines (for example chromafenozide, halofenozide, methoxyfenozide, tebufenozide)

### 9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)
9.2 buprofezin
9.3 cyromazine

### 10. Inhibitors of Oxidative Phosphorylation, ATP Disruptors

10.1 diafenthiuron
10.2 organotins (for example azocyclotin, cyhexatin, fenbutatin-oxide)

### 11. Decouplers of Oxidative Phosphorylation Acting by Interrupting the H-Proton Gradient

11.1 pyrroles (for example chlorfenapyr)
11.2 dinitrophenols (for example binapacryl, dinobuton, dinocap, DNOC)

### 12. Site-I Electron Transport Inhibitors

12.1 METIs (for example fenazaquin, fenpyroximate, pyrimidifen, pyridaben, tebufenpyrad, tolfenpyrad)
12.2 hydramethylnone
12.3 dicofol

### 13. Site-II Electron Transport Inhibitors

13.1 rotenone

14. Site-III Electron Transport Inhibitors
14.1 acequinocyl, fluacrypyrim

### 15. Microbial Disrupters of the Insect Gut Membrane

15.1 Bacillus thuringiensis strains

### 16. Inhibitors of Fat Synthesis

16.1 tetronic acids (for example spirodiclofen, spiromesifen)
16.2 etramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg. No.: 203313-25-1)]

### 17. Arboxamides

(for example flonicamid)

### 18. Ctopaminergic Agonists

(for example amitraz)

### 19. Inhibitors of Magnesium-Stimulated ATPase

(for example propargite)

### 20. Ynaxypyr

### 21. Ereistoxin Analogues

(for example thiocyclam hydrogen oxalate, thiosultap-sodium)

### 22. Biologicals, Hormones or Pheromones

(for example azadirachtin, Bacillus spec., Beauveria spec., Codlemone, Metarrhizium spec., Paecilomyces spec., Thuringiensin, Verticillium spec.)
   23. Active Compounds with Unknown or Unspecific Mechanisms of Action
23.1 umigants (for example aluminium phosphide, methyl bromide, sulphuryl fluoride)
23.2 elective antifeedants (for example cryolite, flonicamid, pymetrozine)
23.3 ite growth inhibitors (for example clofentezine, etoxazole, hexythiazox)
23.4 midoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,
   furthermore the compound 3-methylphenyl propylcarbamate (Tsumacide Z), the compound 3-(5-chloro-3-pyridinyl)-8-(2,2,2-trifluoroethyl)-8-anbicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

The good fungicidal activity of the active compound combinations according to the invention is evident from the example below. While the individual active compounds exhibit weaknesses with regard to the fungicidal activity, the combinations have an activity which exceeds a simple addition of activities.

A synergistic effect of fungicides is always present when the fungicidal activity of the active compound combinations exceeds the total of the activities of the active compounds when applied individually.

The expected activity for a given combination of two active compounds can be calculated as follows (cf. Colby, S. R., “Calculating Synergistic and Antagonistic Responses of Herbicide Combinations”, Weeds 15, pages 20-22, 1967):

If
X is the efficacy, when applying the active compound A at a rate of application of active compound of m g/ha,
Y is the efficacy, when applying the active compound B at a rate of application of active compound of n g/ha,
E is the expected efficacy, when applying the active compounds A and B at rates of application of active compound of m and n g/ha,
   then

$$E = X + Y - \frac{X \cdot Y}{100}$$

The degree of efficacy, expressed in % is denoted. 0% means an efficacy which corresponds to that of the control while an efficacy of 100% means that no disease is observed.

If the actual fungicidal activity exceeds the calculated value, then the activity of the combination is superadditive, i.e. a synergistic effect exists. In this case, the efficacy which was actually observed must be greater than the value for the expected efficacy (E) calculated from the above-mentioned formula.

The invention is illustrated by the following examples

### EXAMPLE A

### Pyricularia oryzae—Test (In Vitro)/Microtest

The microtest was performed in liquid medium with potato-dextrose broth (PDB) using microtitre plates.

The active compound is applied as the technical active substance dissolved in methanol.

A spore suspension of Pyricularia oryzae was used for inoculation. After 4 days of incubation by darkness under shaking (10 Hrz), the optical density in each cavity was evaluated with the aid of a microtitre plate reader.

0% means an efficacy which corresponds to that of the control, while an efficacy of 100% means that no fungal growth is observed.

The table below clearly shows that the observed activity of the active compound combination according to the invention is greater than the calculated activity, i.e. a synergistic effect is present.

TABLE
Pyricularia oryzae-Test (in vitro)/Microtest

| | Rate of application | |
|---|---|---|
| Active compound | of active compound | Efficacy |
| Known: | in ppm | in % |
| Trifloxystrobin | 0.3 | 56 |
| Formononetin | 0.3 | 11 |

Inventive Compound combination:

| | | Rate of | | |
|---|---|---|---|---|
| | | application of | | Expected value, |
| | Ratio | active | Actual | calculated |
| | of the | compound | Efficacy | using Colby's |
| | mixture | in ppm | (%) | formula |
| Trifloxystrobin + | 1:1 | 0.3 + 0.3 | 99 | 61 |
| Formononetin | } | } | | |

### EXAMPLE B

### Ustilago avenae—Test (In Vitro)/Microtest

The microtest was performed in liquid medium with potato-dextrose broth (PDB) using microtitre plates.

The active compound is applied as the technical active substance dissolved in methanol.

A spore suspension of Ustilago avenae was used for inoculation. After 4 days of incubation by darkness under shaking (10 Hrz), the optical density in each cavity was evaluated with the aid of a microtitre plate reader.

0% means an efficacy which corresponds to that of the control, while an efficacy of 100% means that no fungal growth is observed.

The table below clearly shows that the observed activity of the active compound combination according to the invention is greater than the calculated activity, i.e. a synergistic effect is present.

TABLE
Ustilago avenae -Test (in vitro)/Microtest

| Active compound | Rate of application of | |
|---|---|---|
| Known: | active compound in ppm | Efficacy in % |
| BYF 14182 | 0.01 | 95 |
| Formononetin | 0.01 | 13 |

Inventive Compound combination:

| | | | Rate of | | | |
|---|---|---|---|---|---|---|
| | | | application | | | Expected value, |
| | | Ratio | of active | | Actual | calculated |
| | | of the | compound | | Efficacy | using Colby's |
| | | mixture | in ppm | | (%) | formula |
| BYF 14182 + | } | 1:1 | 0.01 + 0.01 | } | 98 | 95 |
| Formononetin | | | | | | |

### EXAMPLE C

### Phytophthora cryptogea—Test (In Vitro)/Microtest

The microtest was performed in liquid medium with potato-dextrose broth (PDB) using microtitre plates.

The active compound is applied as the technical active substance dissolved in methanol.

A mycelium suspension of Phytophthora ayptogea was used for inoculation. After 3 days of incubation by darkness under shaking (10 Hrz), the optical density in each cavity was evaluated with the aid of a microtitre plate reader.

0% means an efficacy which corresponds to that of the control, while an efficacy of 100% means that no fungal growth is observed.

The table below clearly shows that the observed activity of the active compound combination according to the invention is greater than the calculated activity, i.e. a synergistic effect is present.

TABLE
Phytophthora cryptogea -Test (in vitro)/Microtest

| Active compound | Rate of application of | |
|---|---|---|
| Known: | active compound in ppm | Efficacy in % |
| Metalaxyl | 0.3 | 48 |
| Formononetin | 0.3 | 14 |

Inventive Compound combination:

| | | | Rate of | | | |
|---|---|---|---|---|---|---|
| | | | application | | | Expected value, |
| | | Ratio | of active | | Actual | calculated |
| | | of the | compound | | Efficacy | using Colby's |
| | | mixture | in ppm | | (%) | formula |
| Metalaxyl + | } | 1:1 | 0.3 + 0.3 | } | 61 | 55 |
| Formononetin | | | | | | |

### EXAMPLE D

### Ustilago avenae—Test (In Vitro)/Microtest

The microtest was performed in liquid medium with potato-dextrose broth (PDB) using microtitre plates.

The active compound is applied as the technical active substance dissolved in methanol.

A spore suspension of Ustilago avenae was used for inoculation. After 4 days of incubation by darkness under shaking (10 Hrz), the optical density in each cavity was evaluated with the aid of a microtitre plate reader.

0% means an efficacy which corresponds to that of the control, while an efficacy of 100% means that no fungal growth is observed.

The table below clearly shows that the observed activity of the active compound combination according to the invention is greater than the calculated activity, i.e. a synergistic effect is present.

TABLE
Ustilago avenae -Test (in vitro)/Microtest

| Active compound | Rate of application of | |
|---|---|---|
| Known: | active compound in ppm | Efficacy in % |
| Tebuconazole | 0.1 | 96 |
| Formononetin | 0.1 | 17 |

Inventive Compound combination:

| | | | Rate of | | | |
|---|---|---|---|---|---|---|
| | | | application | | | Expected value, |
| | | Ratio | of active | | Actual | calculated |
| | | of the | compound | | Efficacy | using Colby's |
| | | mixture | in ppm | | (%) | formula |
| Tebuconazole + | } | 1:1 | 0.1 + 0.1 | } | 100 | 97 |
| Formononetin | | | | | | |

1. An active compound combination, comprising formononetin of formula (I) 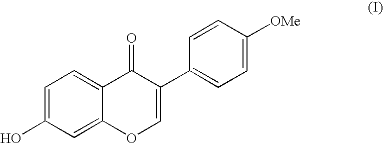
and at least one active compound selected from groups (2) to (24) below:
Group (2) Strobilurins of the general formula (II) 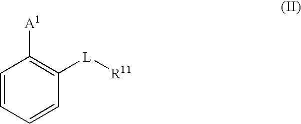
in which
A¹ represents one of the groups 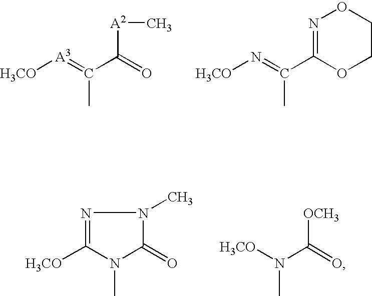
A² represents NH or O,
A³ represents N or CH,
L represents one of the groups 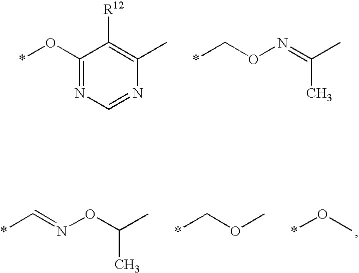
where the bond marked with an asterisk (*) is attached to the phenyl ring,
R¹¹ represents phenyl, phenoxy or pyridinyl, each of which is optionally mono- or disubstituted by identical or different substituents selected from the group consisting of chlorine, cyano, methyl and trifluoromethyl, or represents 1-(4-chlorophenyl)-pyrazol-3-yl or represents 1,2-propanedione-bis(O-methyloxime)-1-yl,
R¹² represents hydrogen or fluorine;
Group (3) Triazoles of the general formula (III) 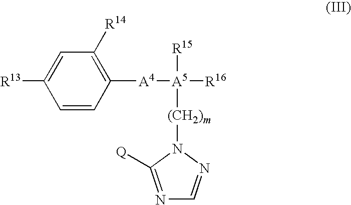
in which
Q represents hydrogen or SH,
m represents 0 or 1,
R¹³ represents hydrogen, fluorine, chlorine, phenyl or 4-chlorophenoxy,
R¹⁴ represents hydrogen or chlorine,
A⁴ represents a direct bond, —CH₂—, —(CH₂)₂— or —O—,
A⁴ furthermore represents *—CH₂—CHR¹⁷— or *—CH═CR¹⁷—, where the bond marked with * is attached to the phenyl ring, in which case R¹⁵ and R¹⁷ together represent —CH₂—CH₂—CH[CH(CH₃)₂]— or —CH₂—CH₂—C(CH₃)₂—,
A⁵ represents C or Si (silicon), or
A⁴ further represents —N(R¹⁷)— and A⁵ furthermore together with R¹⁵ and R¹⁶ represents the group C═N—R¹⁸, in which case R¹⁷ and R¹⁸ together represent the group 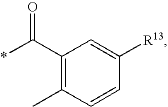
where the bond marked with * is attached to R¹⁷,
R¹⁵ represents hydrogen, hydroxyl or cyano,
R¹⁶ represents 1-cyclopropylethyl, 1-chlorocyclopropyl, C₁-C₄-alkyl, C₁-C₆-hydroxyalkyl, C₁-C₄-alkylcarbonyl, C₁-C₂-haloalkoxy-C₁-C₂-alkyl, trimethylsilyl-C₁-C₂-alkyl, monofluorophenyl or phenyl, or
R¹⁵ and R¹⁶ together represent —O—CH₂—CH(R¹⁸)—O—, —O—CH₂—CH(R¹⁸)—CH₂—, or —O—CH-(2-chlorophenyl)-,
R¹⁸ represents hydrogen, C₁-C₄-alkyl or bromine;
Group (4) Sulphenamides of the general formula (IV) 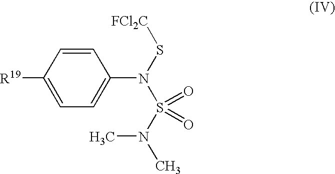
in which R¹⁹ represents hydrogen or methyl;
Group (5) Valinamides selected from the group consisting of
(5-1) iprovalicarb
(5-2) N¹-[2-(4-{[3-(4-chlorophenyl)-2-propynyl]oxy}-3-methoxyphenyl)ethyl]-N²-(methylsulphonyl)-D-valinamide, and
(5-3) benthiavalicarb;
Group (6) Carboxamides of the general formula (V) 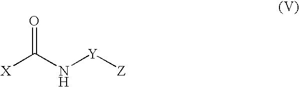
in which
X represents 2-chloro-3-pyridinyl, represents 1-methylpyrazol-4-yl which is substituted in the 3-position by methyl or trifluoromethyl and in the 5-position by hydrogen or chlorine, represents 4-ethyl-2-ethylamino-1,3-thiazol-5-yl, represents 1-methyl-cyclohexyl, represents 2,2-dichloro-1-ethyl-3-methylcyclopropyl, represents 2-fluoro-2-propyl or represents phenyl which is mono- to trisubstituted by identical or different substituents selected from the group consisting of chlorine, methyl, and trifluoromethyl, or
X represents 3,4-dichloroisothiazol-5-yl, 5,6-dihydro-2-methyl-1,4-oxathiin-3-yl, 4-methyl-1,2,3-thiadiazol-5-yl, 4,5-dimethyl-2-trimethylsilylthiophen-3-yl, 1-methylpyrrol-3-yl which is substituted in the 4-position by methyl or trifluoromethyl and in the 5-position by hydrogen or chlorine,
Y represents a direct bond, C₁-C₆-alkanediyl (alkylene) which is optionally substituted by chlorine, cyano or oxo or represents thiophenediyl, or
Y represents C₂-C₆-alkenediyl (alkenylene),
Z represents hydrogen or the group 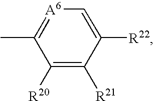
or
Z represents C₁-C₆-alkyl,
A⁶ represents CH or N,
R²⁰ represents hydrogen, chlorine, phenyl which is optionally mono- or disubstituted by identical or different substituents selected from the group consisting of chlorine and di(C₁-C₃-alkyl)aminocarbonyl, or
R²⁰ represents cyano or C₁-C₆-alkyl,
R²¹ represents hydrogen, chlorine, or 1-methylethoxy,
R²² represents hydrogen, chlorine, hydroxyl, methyl or trifluoromethyl, or
R²² represents di(C₁-C₃-alkyl)aminocarbonyl, or
R²⁰ and R²¹ furthermore together represent *—CH(CH₃)—CH₂—C(CH₃)₂— or *—CH(CH₃)—O—C(CH₃)₂— where the bond marked with * is attached to R²⁰;
or the general formula (Va) 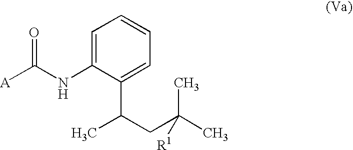
in which
R¹ represents hydrogen, halogen, C₁-C₃-alkyl or C₁-C₃-haloalkyl having 1 to 7 fluorine, chlorine, or bromine atoms, or combinations thereof,
A represents one of the radicals A1 to A8 below: 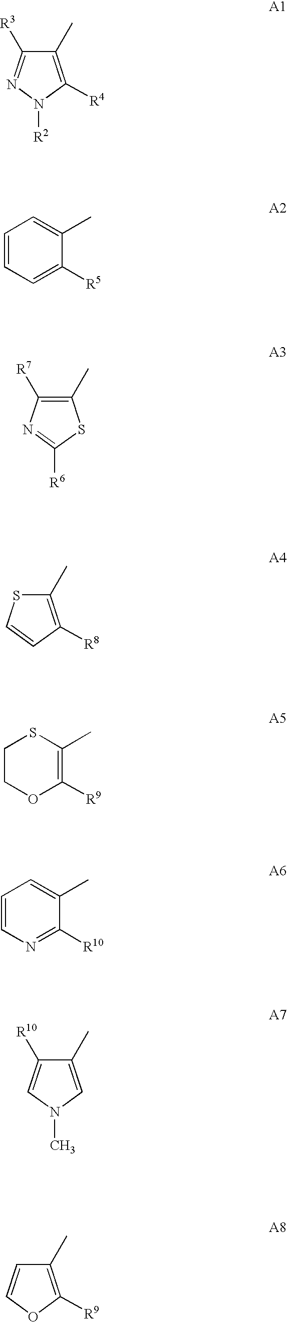
R² represents C₁-C₃-alkyl,
R³ represents hydrogen, halogen, C₁-C₃-alkyl or C₁-C₃-haloalkyl having 1 to 7 fluorine, chlorine, or bromine atoms, or combinations thereof,
R⁴ represents hydrogen, halogen or C₁-C₃-alkyl,
R⁵ represents halogen, C₁-C₃-alkyl or C₁-C₃-haloalkyl having 1 to 7 fluorine, chlorine, or bromine atoms, or combinations thereof,
R⁶ represents hydrogen, halogen, C₁-C₃-alkyl, amino, mono- or di(C₁-C₃-alkyl)amino,
R⁷ represents hydrogen, halogen, C₁-C₃-alkyl or C₁-C₃-haloalkyl having 1 to 7 fluorine, chlorine, or bromine atoms, or combinations thereof,
R⁸ represents halogen, C₁-C₃-alkyl or C₁-C₃-haloalkyl having 1 to 7 fluorine, chlorine, or bromine atoms, or combinations thereof,
R⁹ represents halogen, C₁-C₃-alkyl or C₁-C₃-haloalkyl having 1 to 7 fluorine, chlorine, or bromine atoms, or combinations thereof,
R¹⁰ represents hydrogen, halogen, C₁-C₃-alkyl or C₁-C₃-haloalkyl having 1 to 7 fluorine, chlorine, or bromine atoms, or combinations thereof;
Group (7) Dithiocarbamates selected from the group consisting of
(7-1) mancozeb
(7-2) maneb,
(7-3) metiram,
(7-4) propineb
(7-5) thiram,
(7-6) zineb, and
(7-7) ziram,
Group (8) Acylalanines of the general formula (VI) 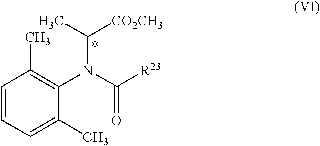
in which
marks a carbon atom in the R or the S configuration,
R²³ represents benzyl, furyl or methoxymethyl;
Group (9): Anilinopyrimidines of the general formula (VII) 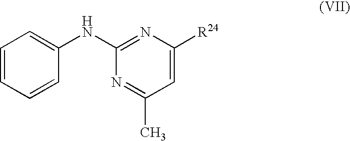
in which
R²⁴ represents methyl, cyclopropyl or 1-propynyl;
Group (10): Benzimidazoles of the general formula (VIII) 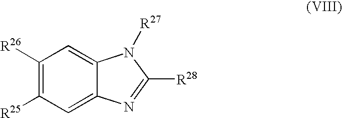
in which
R²⁵ and R²⁶ each represent hydrogen or together represent —O—CF₂—O—,
R²⁷ represents hydrogen, C₁-C₄-alkylaminocarbonyl or represents 3,5-dimethylisoxazol-4-ylsulphonyl,
R²⁸ represents chlorine, methoxycarbonylamino, chlorophenyl, furyl or thiazolyl;
Group (11): Carbamates of the general formula (IX) 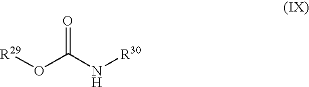
in which
R²⁹ represents n- or isopropyl,
R³⁰ represents di(C₁-C₂-alkyl)amino-C₂-C₄-alkyl or diethoxyphenyl, and salts thereof;
Group (12): Dicarboximides selected from the group consisting of
(12-1) captafol,
(12-2) captan,
(12-3) folpet,
(12-4) iprodione,
(12-5) procymidone, and
(12-6) vinclozolin;
Group (13): Guanidines selected from the group consisting of
(13-1) dodine,
(13-2) guazatine
(13-3) iminoctadine triacetate, and
(13-4) iminoctadine tris(albesilate);
Group (14): Imidazoles selected from the group consisting of
(14-1) cyazofamid,
(14-2) prochloraz,
(14-3) triazoxide, and
(14-4) pefurazoate;
Group (15): Morpholines of the general formula (X) 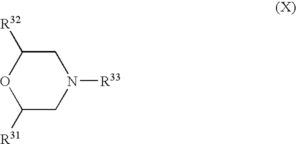
in which
R³¹ and R³² independently of one another represent hydrogen or methyl,
R³³ represents C₁-C₁₄-alkyl, C₅-C₁₂-cycloalkyl, phenyl-C₁-C₄-alkyl, which may be substituted in the phenyl moiety by halogen or C₁-C₄-alkyl or represents acrylyl which is substituted by chlorophenyl or dimethoxyphenyl;
Group (16): Pyrroles of the general formula (XI) 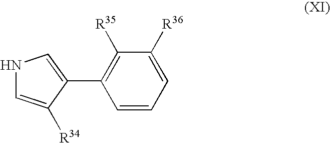
in which
R³⁴ represents chlorine or cyano,
R³⁵ represents chlorine or nitro,
R³⁶ represents chlorine, or
R³⁵ and R³⁶ together represent —O—CF₂—O—;
Group (17): Phosphonates selected from the group consisting of
(17-1) fosetyl-A1, and
(17-2) phosphonic acid;
Group (18): Phenylethanamides of the general formula (XII) 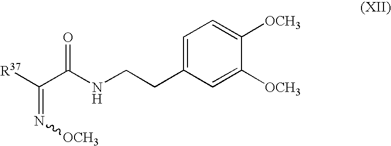
in which
R³⁷ represents unsubstituted or fluorine-, chlorine-, bromine-, methyl- or ethyl-substituted phenyl, 2-naphthyl, 1,2,3,4-tetrahydronaphthyl or indanyl;
Group (19): Fungicides selected from the group consisting of
(19-1) acibenzolar-S-methyl)
(19-2) chlorothalonil,
(19-3) cymoxanil,
(19-4) edifenphos,
(19-5) famoxadone,
(19-6) fluazinam,
(19-7) copper oxychloride,
(19-8) copper hydroxide,
(19-9) oxadixyl,
(19-10) spiroxamine,
(19-11) dithianon,
(19-12) metrafenone,
(19-13) fenamidone,
(19-14) 2,3-dibutyl-6-chlorothieno[2,3-d]pyrimidin-4(3H)-one,
(19-15) probenazole,
(19-16) isoprothiolane,
(19-17) kasugamycin,
(19-18) phthalide,
(19-19) ferimzone,
(19-20) tricyclazole,
(19-21) N-({4-[(cyclopropylamino)carbonyl]phenyl}sulphonyl)-2-methoxybenzamide,
(19-22) 2-(4-chlorophenyl)-N-{2-[3-methoxy-4-(prop-2-yn-1-yloxy)phenyl]ethyl}-2-(prop-2-yn-1-yloxy)acetamide,
(19-23) Diclomezine,
(19-24) Hymexazole,
(19-25) Iprobenfos, and
(19-26) Triflumizole,
Group (20): (Thio)urea derivatives selected from the group consisting of
(20-1) pencycuron,
(20-2) thiophanate-methyl, and
(20-3) thiophanate-ethyl;
Group (21): Amides of the general formula (XIII) 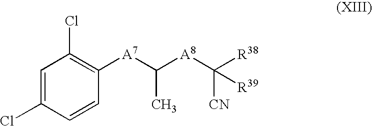
in which
A⁷ represents a direct bond or —O—,
A⁸ represents —C(═O)NH— or —NHC(═O)—,
R³⁸ represents hydrogen or C₁-C₄-alkyl,
R³⁹ represents C₁-C₆-alkyl;
Group (22): Triazolopyrimidines of the general formula (XIV) 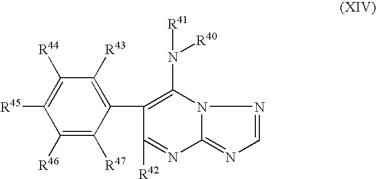
in which
R⁴⁰ represents C₁-C₆-alkyl or C₂-C₆-alkenyl,
R⁴¹ represents C₁-C₆-alkyl, or
R⁴⁰ and R⁴¹ together represent C₄-C₅-alkanediyl (alkylene) which is mono- or disubstituted by C₁-C₆-alkyl,
R⁴² represents bromine or chlorine,
R⁴³ and R⁴⁷ independently of one another represent hydrogen, fluorine, chlorine or methyl,
R⁴⁴ and R⁴⁶ independently of one another represent hydrogen or fluorine,
R⁴⁵ represents hydrogen, fluorine or methyl,
Group (23): Iodochromones of the general formula (XV) 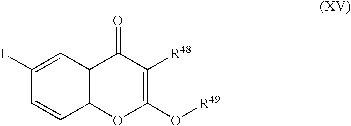
in which
R⁴⁸ represents C₁-C₆-alkyl,
R⁴⁹ represents C₁-C₆-alkyl, C₂-C₆-alkenyl or C₂-C₆-alkynyl;
Group (24): Biphenylcarboxamides of the general formula (XVI) 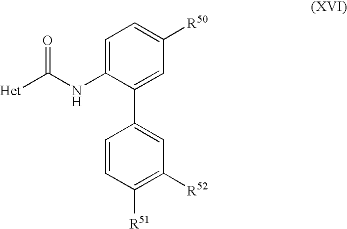
in which
R⁵⁰ represents hydrogen or fluorine,
R⁵¹ represents fluorine, chlorine, bromine, methyl, trifluoromethyl, trifluoromethoxy, —CH═N—OMe or —C(Me)=N—OMe,
R⁵² represents hydrogen, fluorine, chlorine, bromine, methyl or trifluoromethyl,
Het represents one of the radicals Het1 to Het7 below: 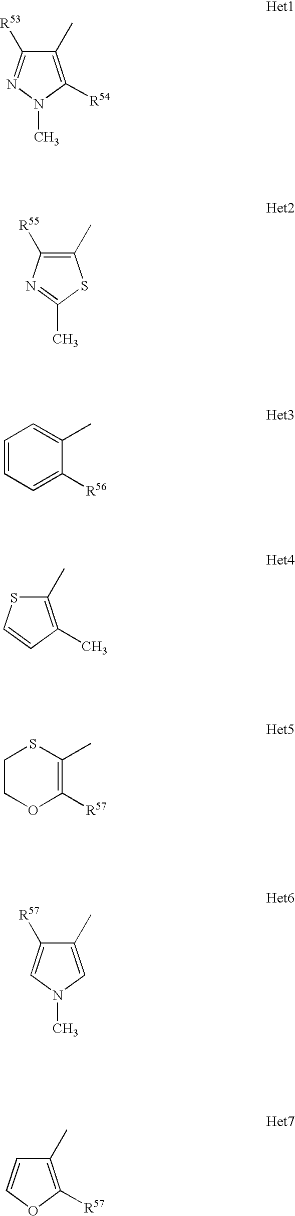
R⁵³ represents iodine, methyl, difluoromethyl or trifluoromethyl,
R⁵⁴ represents hydrogen, fluorine, chlorine or methyl,
R⁵⁵ represents methyl, difluoromethyl or trifluoromethyl,
R⁵⁶ represents chlorine, bromine, iodine, methyl, difluoromethyl or trifluoromethyl,
R⁵⁷ represents methyl or trifluoromethyl.

2. An active compound combination according to claim 1, where the active compounds of groups (2) to (24) are selected from group consisting of:
(2-1) azoxystrobin,
(2-2) fluoxastrobin,
(2-3) (2E)-2-(2-{[6-(3-chloro-2-methylphenoxy)-5-fluoro-4-pyrimidinyl]oxy}phenyl)-2-(methoxyimino)-N-methylethanamide,
(2-4) trifloxystrobin,
(2-5) (2E)-2-(methoxyimino)-N-methyl-2-(2-{[({(1E)-1-[3-(trifluoromethyl)phenyl]ethyliden}amino)oxy]methyl}phenyl)ethanamide,
(2-6) (2E)-2-(methoxyimino)-N-methyl-2-{2-[(E)-({1-[3-(trifluoromethyl)phenyl]ethoxy}imino)methyl]phenyl}ethanamide,
(2-7) orysastrobin,
(2-8) 5-methoxy-2-methyl-4-(2-{[({(1E)-1-[3-(trifluoromethyl)phenyl]ethyliden}amino)oxy]methyl}phenyl)-2,4-dihydro-3H-1,2,4-triazol-3-one,
(2-9) kresoxim-methyl,
(2-10) dimoxystrobin,
(2-11) picoxystrobin,
(2-12) pyraclostrobin,
(2-13) metominostrobin,
(3-1) azaconazole,
(3-2) etaconazole,
(3-3) propiconazole,
(3-4) difenoconazole,
(3-5) bromuconazole,
(3-6) cyproconazole,
(3-7) hexaconazole,
(3-8) penconazole,
(3-9) myclobutanil,
(3-10) tetraconazole,
(3-11) flutriafol,
(3-12) epoxiconazole,
(3-13) flusilazole,
(3-14) simeconazole,
(3-15) prothioconazole,
(3-16) fenbuconazole,
(3-17) tebuconazole,
(3-18) ipconazole,
(3-19) metconazole,
(3-20) triticonazole,
(3-21) bitertanol,
(3-22) triadimenol,
(3-23) triadimefon,
(3-24) fluquinconazole,
(3-25) quinconazole,
(4-1) dichlofluanid,
(4-2) tolylfluanid,
(5-1) iprovalicarb,
(5-3) benthiavalicarb,
(6-1) 2-chloro-N-(1,1,3-trimethylindan-4-yl)nicotinamide,
(6-2) boscalid,
(6-3) furametpyr,
(6-4) N-(3-p-tolylthiophene-2-yl)-1-methyl-3-trifluoromethyl-1H-pyrazole-4-carboxamide,
(6-5) ethaboxam,
(6-6) fenhexamid,
(6-7) carpropamid,
(6-8) 2-chloro-4-(2-fluoro-2-methylpropionylamino)-N,N-dimethylbenzamide,
(6-9) picobenzamid,
(6-10) zoxamide,
(6-11) 3,4-dichloro-N-(2-cyanophenyl)isothiazole-5-carboxamide,
(6-12) carboxin,
(6-13) tiadinil,
(6-14) penthiopyrad,
(6-15) silthiofam,
(6-16) N-[2-(1,3-dimethylbutyl)phenyl]-1-methyl-4-(trifluoromethyl)-1H-pyrrole-3-carboxamide,
(6-17) N-{2-[3-chloro-5-(trifluoromethyl)pyridin-2-yl]ethyl}-2-(trifluoromethyl)benzamide,
(6a-8) 5-fluoro-1,3-dimethyl-N-[2-(1,3,3-trimethylbutyl)phenyl]-1H-pyrazole-4-carboxamide,
(6a-2) N-[2-(1,3-dimethylbutyl)phenyl]-5-fluoro-1,3-dimethyl-1H-pyrazole-4-carboxamide,
(6a-16) N-[2-(1,3-dimethylbutyl)phenyl]-2-(trifluoromethyl)benzamide,
(6a-13) N-[2-(1,3-dimethylbutyl)phenyl]-2-iodobenzamide,
(6b-2) N-(2-[1,1′-bicyclopropyl]-2-ylphenyl)-3-(difluoromethyl)-1-methyl-1H-pyrazole-4-carboxamide,
(7-1) mancozeb,
(7-2) maneb,
(7-3) metiram,
(7-4) propineb,
(7-5) thiram,
(7-6) zineb,
(7-7) ziram,
(8-1) benalaxyl,
(8-2) furalaxyl,
(8-3) metalaxyl,
(8-4) metalaxyl-M,
(8-5) benalaxyl-M,
(9-1) cyprodinil,
(9-2) mepanipyrim,
(9-3) pyrimethanil,
(10-1) 6-chloro-5-[(3,5-dimethylisoxazol-4-yl)sulphonyl]-2,2-difluoro-5H-[1,3]dioxolo[4,5-f]benzimidazole,
(10-2) benomyl,
(10-3) carbendazim,
(10-4) chlorfenazole,
(10-5) fuberidazole,
(10-6) thiabendazole,
(11-1) diethofencarb,
(11-2) propamocarb,
(11-3) propamocarb-hydrochloride,
(11-4) propamocarb-fosetyl,
(12-1) captafol,
(12-2) captan
(12-3) folpet,
(12-4) iprodione
(12-5) procymidone,
(12-6) vinclozolin
(13-1) dodine,
(13-2) guazatine,
(13-3) iminoctadine triacetate,
(14-1) cyazofamid,
(14-2) prochloraz,
(14-3) triazoxide,
(14-4) pefurazoate
(15-1) aldimorph,
(15-2) tridemorph,
(15-3) dodemorph,
(15-4) fenpropimorph,
(15-5) dimethomorph,
(16-1) fenpiclonil,
(16-2) fludioxonil,
(16-3) pyrrolnitrin,
(17-1) fosetyl-A1,
(17-2) phosphonic
(18-1) 2-(2,3-dihydro-1H-inden-5-yl)-N-[2-(3,4-dimethoxyphenyl)ethyl]-2-(methoxyimino)acetamide,
(18-2) N-[2-(3,4-dimethoxyphenyl)ethyl]-2-(methoxyimino)-2-(5,6,7,8-tetrahydronaphthalen-2-yl)acetamide,
(18-3) 2-(4-chlorophenyl)-N-[2-(3,4-dimethoxyphenyl)ethyl]-2-(methoxyimino)acetamide,
(18-4) 2-(4-bromophenyl)-N-[2-(3,4-dimethoxyphenyl)ethyl]-2-(methoxyimino)acetamide,
(18-5) 2-(4-methylphenyl)-N-[2-(3,4-dimethoxyphenyl)ethyl]-2-(methoxyimino)acetamide,
(18-6) 2-(4-ethylphenyl)-N-[2-(3,4-dimethoxyphenyl)ethyl]-2-(methoxyimino)acetamide,
(19-1) acibenzolar-S-methyl,
(19-2) chlorothalonil,
(19-3) cymoxanil,
(19-4) edifenphos,
(19-5) famoxadone,
(19-6) fluazinam,
(19-7) copper oxychloride,
(19-9) oxadixyl,
(19-10) spiroxamine,
(19-11) dithianon,
(19-12) metrafenone,
(19-13) fenamidone,
(19-14) 2,3-dibutyl-6-chlorothieno[2,3-d]pyrimidin-4(3H)-one,
(19-15) probenazole,
(19-16) isoprothiolane,
(19-17) kasugamycin,
(19-18) phthalide,
(19-19) ferimzone,
(19-20) tricyclazole,
(19-21) N-({4-[(cyclopropylamino)carbonyl]phenyl}sulphonyl)-2-methoxybenzamide,
(19-22) 2-(4-chlorophenyl)-N-{2-[3-methoxy-4-(prop-2-yn-1-yloxy)phenyl]ethyl}-2-(prop-2-yn-1-yloxy)acetamide,
(20-1) pencycuron,
(20-2) thiophanate-methyl,
(20-3) thiophanate-ethyl,
(21-1) fenoxanil,
(21-2) diclocymet,
(22-1) 5-chloro-N-[(1S)-2,2,2-trifluoro-1-methylethyl]-6-(2,4,6-trifluorophenyl)[1,2,4]triazolo[1,5-a]pyrimidine-7-amine,
(22-2) 5-chloro-N-[(1R)-1,2-dimethylpropyl]-6-(2,4,6-trifluorophenyl)[1,2,4]triazolo[1,5-a]pyrimidine-7-amine,
(22-3) 5-chloro-6-(2-chloro-6-fluorophenyl)-7-(4-methylpiperidin-1-yl)[1,2,4]triazolo[1,5-a]pyrimidine,
(22-4) 5-chloro-6-(2,4,6-trifluorophenyl)-7-(4-methylpiperidin-1-yl)[1,2,4]triazolo[1,5-a]pyrimidine
(23-1) 2-butoxy-6-iodo-3-propylbenzopyran-4-one,
(23-2) 2-ethoxy-6-iodo-3-propylbenzopyran-4-one,
(23-3) 6-iodo-2-propoxy-3-propylbenzopyran-4-one,
(23-4) 2-but-2-ynyloxy-6-iodo-3-propylbenzopyran-4-one
(23-5) 6-iodo-2-(1-methylbutoxy)-3-propylbenzopyran-4-one,
(23-6) 2-but-3-enyloxy-6-iodobenzopyran-4-one,
(23-7) 3-butyl-6-iodo-2-isopropoxybenzopyran-4-one,
(24-1) N-(3′,4′-dichloro-5-fluoro-1,1′-biphenyl-2-yl)-3-(difluoromethyl)-1-methyl-1H-pyrazole-4-carboxamide,
(24-2) 3-(difluoromethyl)-N-{3′-fluoro-4′-[(E)-(methoxyimino)methyl]-1,1′-biphenyl-2-yl}-1-methyl-1H-pyrazole-4-carboxamide,
(24-3) 3-(trifluoromethyl)-N-{3′-fluoro-4′-[(E)-(methoxyimino)methyl]-1,1′-biphenyl-2-yl}-1-methyl-1H-pyrazole-4-carboxamide,
(24-4) N-(3′,4′-dichloro-1,1′-biphenyl-2-yl)-5-fluoro-1,3-dimethyl-1H-pyrazole-4-carboxamide,
(24-5) N-(4′-chloro-3′-fluoro-1,1′-biphenyl-2-yl)-2-methyl-4-(trifluoromethyl)-1,3-thiazole-5-carboxamide,
(24-6) N-(4′-chloro-1,1′-biphenyl-2-yl)-4-(difluoromethyl)-2-methyl-1,3-thiazole-5-carboxamide
(24-7) N-(4′-bromo-1,1′-biphenyl-2-yl)-4-(difluoromethyl)-2-methyl-1,3-thiazole-5-carboxamide and
(24-8) 4-(difluoromethyl)-2-methyl-N-[4′-(trifluoromethyl)-1,1′-biphenyl-2-yl]-1,3-thiazole-5-carboxamide.

3. (canceled)

4. A method for controlling phytopathogenic fungi on seed, comprising treating said seed by applying an active compound combination according to claim 1 to the seed.

5. A method for controlling phytopathogenic fungi on transgenic plants, comprising treating said transgenic plants by applying an active compound combination according to claim 1 to said transgenic plants.

6. A method for controlling phytopathogenic fungi on seed of transgenic plants, comprising treating said seed of transgenic plants by applying an active compound combination according to claim 1 to the seed of transgenic plants.

7. Seed that has been treated with an active compound combination according to claim 1, wherein the formononetin and a fungicide selected from groups (2) to (24), have been applied either simultaneously or separately.

8. A method for controlling unwanted phytopathogenic fungi, comprising applying an active compound combination according to claim 1 to the unwanted phytopathogenic fungi, their habitat, seed, or combinations thereof.

9. A process for preparing a fungicidal composition, comprising mixing an active compound combination according to claim 1 with an extender, a surfactant or combinations thereof.

10. A method of coating or incubating a seed with an active compound combination, comprising applying the formononetin and a fungicide selected from groups (2) to (24) of claim 1 to said seed, at the same time.

11. A method of coating or incubating a seed with an active compound combination, comprising applying separately the formononetin and a fungicide selected from groups (2) to (24) of claim 1 to said seed, optionally with at least one further separation layer between active ingredient layers.
